(19) United States
(12) Patent Application Publication
(10) Pub. No.: US 2008/0187912 A1
(43) Pub. Date: Aug. 7, 2008

(54) Oligonucleotide arrays for high resolution HLA typing

(75) Inventors: Effie W. Petersdorf, Seattle, WA (US); Zhen Guo, Bellevue, WA (US); John A. Hansen, Mercer Island, WA (US); Leroy Hood, Seattle, WA (US)

Correspondence Address: MYERS BIGEL SIBLEY & SAJOVEC, PO BOX 37428, RALEIGH, NC, 27627, US

(73) Assignees: Fred Hutchinson Cancer Research Center, Seattle, WA (US); The University of Washington Office of Technology Transfer, Seattle, WA (US)

(21) Appl. No.: 11/517,956

(22) Filed: Sep. 7, 2006

Related U.S. Application Data
(62) Division of application No. 10/018,112, filed on Oct. 28, 2002.
(60) Provisional application No. 60/139,843, filed on Jun. 17, 1999.

(51) Int. Cl. C12Q 1/68

(52) U.S. Cl. 435/6

(57) ABSTRACT

Arrays of HLA Class I oligonucleotide probes on a solid support are provided, wherein the probes are sufficient to represent at least 80% of the known polymorphisms in exons 2 and 3 of the HLA Class I locus.

### CROSS-REFERENCES TO RELATED APPLICATIONS

This application claims the benefit of U.S. Provisional Patent Application Ser. No. 60/139,843, filed on Jun. 17, 1999.

### STATEMENT AS TO RIGHTS TO INVENTIONS MADE UNDER FEDERALLY SPONSORED RESEARCH AND DEVELOPMENT

The U.S. government may have certain rights in the invention pursuant to Grant No. CA 18029, R01 HG01713-02 and 98-3300-416457 received from the U.S. National Institutes of Health.

### FIELD OF THE INVENTION

This invention relates to arrays of oligonucleotides that are useful for HLA typing. The arrays are specifically designed and constructed to facilitate diagnostic evaluations and assist in phenotypic analysis for such uses as donor/recipient transplant compatibility.

### BACKGROUND OF THE INVENTION

Oligonucleotide array technology is a revolutionary tool in modern molecular biology. The combination of a standard nucleic acid hybridization approach with innovative high-density DNA array technology has proven to be a powerful method for high-throughput DNA sequence analysis. Initially developed to improve sequencing efforts in the Human Genome Project, the oligonucleotide array technology has been successfully applied to many fields of molecular biology, including large scale gene discovery, monitoring the expression of thousands of genes, mutation and polymorphism detection, as well as mapping of genomic clones.

Oligonucleotide arrays are manufactured either by in situ combinatorial oligonucleotide synthesis or by conventional synthesis followed by on-chip immobilization of the oligonucleotide onto the solid support. Sample DNA is amplified by the polymerase chain reaction (PCR), labeled with a fluorescent tag and hybridized to the oligonucleotide array. The hybridization pattern is measured by fluorescence scanning and the intensity of each hybridization signal is quantified using a “spot-finding” software.

Oligonucleotide arrays provide the ability to assay many different combinations of DNA sequences simultaneously. Oligonucleotide arrays have been applied to study diverse and complex genetic systems. The utility of array technology for the detection of new mutations and polymorphisms, gene discovery, gene expression and mapping has been convincingly demonstrated (Pease et al., Proc. Natl. Acad. Sci. USA 91:5022-5026 (1994); Wodicka et al., Nature Biotech., 15:1359-1367 (1997); Hacia et al., Nature Genetics 18:155-158 (1998); Sapolsky and Lipshutz, Genomics 33: 445-456 (1996)). However, an efficient HLA array has not yet been produced.

Thus while the innovative high-density oligonucleotide array technology has proven to be a powerful method for high-through-put DNA sequence analysis, a practical system to systematically identify all alleles of any HLA gene has never been developed.

The human major histocompatibility genes are among the most polymorphic genes known in the human genome. HLA antigens are encoded by a series of closely linked genes located at the position p21 on chromosome 6. Genes of the HLA region span approximately 4 million bases of DNA, and are clustered into three distinct regions designated class I, class II and class III. Genes within the class I and class II regions share structural and functional properties and are considered to be part of the immunoglobulin gene super family. Although distinct in sequence and structure, both class I and class II genes encode proteins that are critical in controlling T-cell recognition and determining histocompatibility in marrow transplantation (Rammensee, Curr. Opin. Immunol. 7:85-96 (1995)).

At least 17 loci including several pseudogenes exist in the HLA class I region. Three of these loci encode HLA-A, -B and -C alloantigens that constitute the major class I determinants important for matching in tissue transplantation. The HLA-A, -B and -C loci show a striking degree of sequence and structural homology with one and another and genes at all three loci are highly polymorphic (Bodmer et al., Tissue Antigens 49:297-321 (1997)). Currently, more than 86 HLA-A, 185 HLA-B and 45 HLA-C alleles have been described. More recently, three additional class I genes, HLA-E, -F and -G, have been defined (Geraghty et al., J. Exp. Med. 171:1-19; 53, 54 (1990); Geraghty et al., Proc. Natl. Acad. Sci. USA 84:9145-49. 54 (1987); Koller et al., J. Immunol. 141:897-904 (1988)). Although HLA-E, -F and -G genes are structurally homologous with HLA-A, B and C genes, they appear to have limited polymorphism, the tissue expression of their encoded molecules is more restricted, and their potential role as transplantation antigens is unknown.

The HLA class II region is comprised of nine distinct genes: DRA, DRB1, DRB3, DRB4, DRB5, DQA, DQB, DPA and DPB. Six additional class II genes or gene fragments have been described but these are either nonfunctional pseudogenes or do not encode proteins known to participate in transplant-related immune interactions. Class II genes are divided into five families, designated DR, DQ, DO, DN and DP, based on their degree of sequence homology and their location within the HLA-D region. As with class I genes, class II DR, DQ and DP genes show a striking degree of polymorphism, with more than 220 alleles thus far defined at the DRB1 locus (Bodmer et al., supra, (1997)).

The identification of HLA alleles has significance in both medical clinics and genetic research. Detection of HLA polymorphisms and their frequencies in population is the key to study the fundamental issues in immunogenetics, such as evolutionary diversification of HLA system and the linkage between certain HLA types and disease susceptibility.

The clinical importance of the identification of HLA polymorphisms can be illustrated by its application in marrow transplantation. Matching of HLA allele types of patients and those of donors has proven essential for the success of unrelated marrow transplantation for hematologic malignancies. Current standards for HLA typing include serological methods for HLA-A, -B and -C antigens and DNA-based typing for class II HLA-DRB1 and DQB1 alleles. DNA-based methods remain the most accurate when compared to serological methods. The optimization of the outcome of unrelated marrow transplantation thus requires the comprehensive analysis of both HLA class I and class II genes of transplant populations.

The limiting factor in large-scale genetic analysis of transplant populations has been methodologic and directly involves the technical ability to accurately define the alleles of highly polymorphic HLA genes in a cost-effective and efficient manner. Although recent progress in the development of traditional probe hybridization and sequencing-based methods has allowed alleles to be determined with accuracy, large-scale efforts in genetic analysis of transplant populations are hampered by the cost of available methods, particularly for the highly polymorphic HLA-A, -B and -C genes.

There therefore exists a need in the art for methods for the identification of all alleles of any HLA gene. This invention addressed these needs by providing methods and compositions for the systematic identification HLA alleles and for HLA typing.

### SUMMARY OF THE INVENTION

In one aspect, the present invention provides an array of HLA Class I oligonucleotide probes on a solid support, wherein the probes are sufficient to represent at least 80% of the known polymorphisms of the HLA Class I locus. Preferably, the probes represent at least 90%, and more preferably at least 98% of the known polymorphisms of the HLA Class I locus. Particularly preferred probes are those that represent the known polymorphisms of exons 2 and 3 of the HLA Class I locus. Typically the oligonucleotide probes will comprise from about 17 to 23 nucleic acid residues, with those having about 20 nucleic acid residues being preferred. Additionally, the HLA Class I oligonucleotide probes are preferably HLA-A oligonucleotide probes, HLA-B oligonucleotide probes or HLA-C oligonulceotide probes.

The microarray will also typically comprise the probes at a surface density of about 250 to about 450 angstrom²/molecule, preferably about 325 to about 375 angstrom²/molecule.

In another aspect, the present invention provides a method of preparing an array of covalently-attached oligonucleotide probes, the method comprising:

(a) contacting a solid support with an aminoalkyltrialkoxysilane in the vapor phase at reduced pressure to form an aminoalkylsilane-derivatized solid support;

(b) contacting the aminoalkylsilane-derivatized solid support with a linking group to covalently attach the linking group to the aminoalkylsilane-derivatized solid support to form a linking group-modified solid support; and

(c) attaching a plurality of oligonucleotide probes to the linking group-modified solid support to form the array of covalently-attached oligonucleotide probes.

In still another aspect, the present invention provides a method of HLA Class I tissue typing, the method comprising:
(a) amplifying exons 2 and 3 from a genomic sample of tissue using labeled primers and an asymmetric PCR method to form a labeled, single-stranded DNA sample;
(b) contacting, under hybridization conditions, the labeled, single-stranded DNA sample with a microarray prepared by the methods described herein; and
(c) detecting a hybridization pattern for the DNA sample and assigning an HLA Class I allele type by analysis of the hybridization pattern.

In yet another aspect, the present invention provides a method of HLA tissue typing, said method comprising:
(a) selectively amplifying the HLA regions in a genomic sample using asymmetric PCR and labeled primers to form a labeled, single-stranded DNA sample;
(b) contacting under hybridization conditions the labeled, single-stranded DNA sample with an HLA microarray prepared by the methods described herein; and
(c) detecting a hybridization pattern for the DNA sample and assigning an HLA allele type by analysis of the hybridization pattern.

A method of HLA-B tissue typing, said method comprising:
(a) amplifying exons 2 and 3 from a genomic sample of tissue using labeled primers and an asymmetric PCR method to form a labeled, single-stranded DNA sample;
(b) contacting under hybridization conditions, the labeled, single-stranded DNA sample with an HLA-B microarray described herein; and
(c) detecting a hybridization pattern for the DNA sample and assigning an HLA-B allele type by analysis of the hybridization pattern.

### DETAILED DESCRIPTION

The present invention provides compositions and methods for detecting polymorphisms in genes. In particular, the invention provides compositions and methods for determining the genotype of an individual at a particular allele. Within a particularly preferred embodiment, the alleles are alleles in the major histocompatibility locus. Within one embodiment, the alleles are alleles of a HLA Class I gene. Within another embodiment, the alleles are alleles of a HLA Class II gene. Within yet another embodiment, the HLA Class I alleles are alleles of HLA-A, HLA-B or HLA-C.

### Overview of Oligonucleotide Array Technology

Oligonucleotide array technology has become a valuable tool in modern molecular biology. The combination of a standard nucleic acid hybridization approach with innovative high-density DNA array technology has proven to be a powerful method for high-throughput DNA sequence analysis. The technology has been successfully applied to many fields of molecular biology, including large scale gene discovery, monitoring the expression of thousands of genes, mutation and polymorphism detection, as well as mapping of genomic clones.

Solid support-based oligonucleotide arrays can be manufactured either by in situ combinatorial oligonucleotide synthesis or by conventional oligonucleotide synthesis followed by immobilization of the oligonucleotide onto the solid support.

When the arrays are used for sequencing, a sample of DNA is amplified (typically using the polymerase chain reaction (PCR)), labeled with a detectable tag and hybridized to the oligonucleotide array in which the locations of various probe oligonucleotides are known. The hybridization pattern is then measured by, for example, fluorescence scanning, and the intensity of each hybridization signal is quantified using a “spot-finding” software. The pattern provides the practitioner with a sequence for an unknown piece of DNA.

### Synthesis of Oligonucleotide Arrays on Solid Supports

The attachment of nucleic acids or oligonucleotide molecules to solid supports to create highly dense patterns of diverse oligonucleotide probes on a single surface has been demonstrated by, for example, Maskos and Southern (Nuc. Acids. Res. 20:1679-1684 (1992)), Blanchard and Hood (Bioelectronics 11:687-690 (1996)), and Fodor et al. (Science 251:767-773 (1991)). Arrays with as many as 10⁵ different types of oligonucleotide probes on a single silicon or glass surface have been constructed. The fidelity of these arrays has been demonstrated by hybridization of DNA labeled with a fluorescent or radioisotope tag.

Two methodologies have been used to synthesize oligonucleotide arrays. Saiki et al. (Proc. Natl. Acad. Sci. USA. 86:6230-6234 (1989)) and Chrisey et al. (Nuc. Acids Res. 24:3040-3047 (1996)), for example, demonstrated that presynthesized oligonucleotide probes can be delivered to a solid support by high-speed robotics, and then immobilized on the surface. The resolution of the resulting oligonucleotide array is determined by both the spatial resolution of the delivery systems and the physical space requirement of the delivered oligonucleotide solution volume. The surface density of the immobilized oligonucleotides varies greatly with different solid surface and linkage chemistries (Guo, et al., Nuc. Acids Res. 22:5456-5465 (1994); Fahy, et al., Nuc. Acids Res. 21:1819-1826 (1993); Wolf, et al., Nuc. Acids Res. 15:2911-2926 (1987); and Ghosh, et al., Nuc. Acids Res. 15:5353-5372 (1987)).

In another approach oligonucleotide probes are synthesized directly onto the solid support, nucleotide by nucleotide, through a series of coupling and deprotection steps. Both conventional solid-phase oligonucleotide synthesis methods and light-directed combinatorial synthesis methods have been successfully applied in this in situ fabrication process (Fodor et al., supra (1991) and Gilham, Biochemistry 7:2809-2813 (1968)). High reaction yields in both the coupling and the deprotection steps are critical for the success of in situ synthesis. The preparation of in situ arrays can be automated and thereby increase the complexity of the array compared to the use of presynthesized oligonucleotides.

a) Spatially-resolved Attachment Chemistry

Preferably, an oligonucleotide is immobilized onto a solid support through a single covalent bond. Gilham (Biochemistry, 7:2809-2813 (1968)), for example, described the attachment of DNA molecules to paper using carbodiimide via the 5′-end terminal phosphate group. Suitable supports for covalent immobilization of DNA include glass, acrylamide gel, latex particles, controlled pore glass, dextran supports, polystryene matrices and avidin-coated polystyrene beads and have been described (Guo, et al., Nuc. Acids Res. 22:5456-5465 (1994); Fahy, et al., Nuc. Acids Res. 21:1819-1826 (1993); Wolf, et al., Nuc. Acids Res. 15:2911-2926 (1987); Ghosh, et al., Nuc. Acids Res. 15:5353-5372 (1987); Gingeras et al., Nuc Acids Res. 15:5773-5790 (1987); Rasmussen et al., Anal. Biochem. 198:138-142 (1991); and Lund et al., Nuc. Acids Res. 16:10861-10880 (1988)). Several other solid supports, such as nitrocellulose and nylon membranes were employed for oligonucleotide immobilization using UV-activated DNA-surface cross-linking chemistry (Meinkoth and Wahl, Anal. Biochem. 138:267-284 (1984)). However, in these cases, DNA molecules were non-covalently bound to the surface at multiple sites, hampering reproducibility and stability.

Fodor, et al. (supra (1991)) demonstrated the use of photolithographic technology to synthesize high-density oligonucleotide arrays on silicon substrates. In this process, crosslinkers are first made by exposing a photochemically-labile organosilane surface to UV light. The resulting pattern is then reacted with heterobifunctional crosslinking molecules. The oligonucleotide molecules are then bound to these crosslinkers to form a well-defined DNA pattern on the surface. Spatial resolution of 1 micron per DNA spot is feasible using this approach.

Although newer chemistries have greatly improved the density of arrays compared to earlier methods, the capacity of these arrays has been limited chiefly by the two-dimensional nature of the solid surface. Three-dimensional immobilization matrices have been developed to increase capacity. Yershor, et al. (Genetics 93:4913-4918 (1996)), for example, have produced DNA arrays by immobilizing oligonucleotides in acrylamide gel at a density of 20,000 to 30,000 different oligonucleotide probes per cm², two orders of magnitude higher than the capacity of two-dimensional supports. The three-dimensional support permits high oligonucleotide loading and enhanced hybridization. However, because only short oligonucleotides can diffuse into gel matrix, the application of this approach is limited.

b) Spatially Addressable Parallel Chemical Synthesis

Solid phase DNA synthesis can be accomplished with a number of different chemistries. Froehler et al. (Nuc. Acids Res. 14:5399-5407 (1986) and Mcbride and Caruthers (Tetrahedron Lett. 24:245-248 (1983) have demonstrated solid phase DNA synthesis chemistries utilizing H-phosphonate and phosphoramidites, which covalently attach an organic linker molecule to a surface and build the oligonucleotide off the terminus of the linker through successive coupling and deprotection steps.

Based on this scheme, two distinct approaches have been developed to construct surface-bound oligonucleotide arrays. One approach (Fodor, et al., supra (1991)) combines solid-phase DNA synthesis with semiconductor-based photolithography. The major advantage of this approach is the potential to synthesize very high-density arrays comprised of 50 micron spots or less. However, the major drawback to this approach is the need for a photolithographic mask for each unique array of oligonucleotides. For example, an array of 25-mers would require 100 different masks. The expense of synthesizing these arrays is proportional to the number of unique masks.

In a second approach, Southern et al. (supra, 1994) used microfabricated ink-jet pumps, similar to those used in certain ink-jet printers to deliver synthesis reagents onto the surface of a solid support. Within this method, the surface is scanned across a set of ink-jet pumps using a computer-controlled x-y translation stage. In each coupling step, DNA monomers are delivered to the defined area at rates of several hundred drops per second. These in situ approaches permit large numbers of arrays of unlimited combinatorial matrices to be made in fairly few steps.

Each of these in situ approaches permit large numbers of arrays of unlimited combinatorial matrices to be made in fairly few steps.

### Hybridization and Detection

Hybridization of DNA to a solid support has similar thermodynamic behavior compared to hybridization of DNA in solution. The stability of the double helix can be characterized by its melting temperature, which is strongly dependent upon oligonucleotide sequence and composition of the solvent (Wetmur, Crit. Rev. Biochem. & Mol. Bio. 26:227-259 (1991)). This strong-dependence of the duplex stability on oligonucleotide sequence, especially for short oligonucleotides, makes it difficult to design adequately stringent conditions for hybridization with oligonucleotide arrays, which usually vary widely in base composition. Thus, a large number of false positive or negative signals may occur when hybridization is performed on complex oligonucleotide arrays. Several approaches have been employed to eliminate the sequence-dependence of the stability of duplexes. Utilization of tetramethylammonium chloride (TMAC) in the hybridization solution is the most popular approach (Wood, et al., Proc. Natl. Acad. Sci. USA. 82:1585-1588 (1985) and Riccelli and Benight, Nuc. Acids Res. 21:3785-3788 (1993)). TMAC was found to neutralize stability of duplexes imparted by sequences and allow the stringency of hybridization to be controlled as a function of probe length. Similar “isostabilization” function has also been described for other reagents (Rees, et al., Biochemistry 32:137-144 (1993)).

The thermodynamic stability of solid-phase hybridization is also affected by differences between the perfectly matched duplex versus the mismatched duplex, which constitutes the fundamental limitation to sequence-specific recognition in hybridization. The binding of a probe mismatched at a single base is compared with that of a perfectly matched probe; the difference in duplex stability is used to identify the target sequence. In many cases, the differences in stability of a perfect match and a single-base mismatch are so small that discrimination between a perfect match and a single base mismatch cannot be achieved using common hybridization-washing procedures. Guo et al. described an approach to substantially increase the discrimination of single-base mismatches by using artificial mismatches (Guo et al., Nature Biotech. 15:331-335 (1997)). In this approach, an “artificial” mismatch is intentionally inserted into the oligonucleotide probe sequence, and the discrimination compares the stability of two mismatches versus one mismatch. An enhancement of the discrimination, as high as 200% of differential melting temperature, is generally achieved in hybridization with oligonucleotide arrays.

In vitro data indicate that DNA hybridization in free solution and on surfaces is often a reaction rate-limited process. In a study of nitrocellulose membrane arrays, the hybridization kinetics were found to be proportional to the concentration of immobilized DNA. Recently, Chan et al. proposed a mathematical model of hybridization on solid supports (Chan et al., Biophysical J. 69:2243-2255 (1995)). This theory hypothesizes two different mechanisms by which DNA targets can hybridize with immobilized oligonucleotide probes: direct hybridization from solution and hybridization by DNA targets that adsorb nonspecifically on the surface and then diffuse to the probes. The hybridization rate depends strongly on both the DNA diffusion constant in solution and the DNA adsorption/desorption constant on surface.

Nanogen Inc. has developed a practical system to accelerate the hybridization process using electric fields to facilitate the diffusion of DNA targets to the immobilized probes (Sosnowski et al., Proc. Natl. Acad. Sci. USA 94:1119-1123 (1997) and Cheng et al., Nature Biotech. 16:541-546 (1998)). In this system, oligonucleotide arrays are synthesized on the surface of a silicon electrode. DNA molecules, which have a large negative charge, can be moved in an electric field to an area of net positive charge and concentrate significantly on the electrode surface. The concentrating effect accelerates the hybridization of DNA targets. Another advantage of this system is the reversibility of the hybridization in which non-specifically bound DNA target molecules can be easily removed from the oligonucleotide arrays by reversing the polarity of the field.

Detection of Hybridization Events on Solid Supports

Hybridization assays have been greatly simplified by the PCR. PCR can selectively amplify the number of copies of a particular DNA sequence of interest by 6 to 8 orders of magnitude (Saiki et al., Science 239:487-491 (1988)). This amplification process, together with the high density of oligonucleotide arrays available with the solid-phase synthesis, makes it much easier to generate detectable hybridization signals on a solid surface. As a result of this increased hybridization signal intensity, nonradioactive detection methods are increasingly preferred for oligonucleotide arrays, especially in clinical applications of large numbers of DNA samples.

These advances notwithstanding, the two dimensional nature of oligonucleotide arrays and the need for extensive washing steps to remove mismatched and nonspecifically bound DNA target greatly limit the hybridization signal intensity. Several detection methods are currently available of which the fluorescence-based methods are the most popular. Quantitative hybridization data available from these methods affords the advantages of rapid image analysis, direct comparison and digital archiving. Both the intensity of the fluorescent signal and the background depend strongly on environmental factors, such as dryness of the surface and the support materials (Guo et al., supra (1994)). The influence of environmental factors to the strength of the signal and background mandates stringency for conditions and often precludes the use of highly fluorescent supports, such as nylon membranes.

In the analysis of complex genome systems, the use of multiple fluorescent dyes to simultaneously distinguish different DNA molecules is an important methodologic advancement. DNA targets in hybridization systems can be fluorescently labeled either directly or indirectly. The direct fluorescent label systems for DNA molecules include derivatives of fluorescein and rhodamine dyes, which can be easily attached to the end of DNA strand.

Biotin is the most commonly used indirect fluorescent label. Biotin can be easily incorporated into DNA molecules and detected using avidin or streptavidin by a covalently linked reporter group, such as alkaline phospatase and horseradish peroxidase (Rees and Kurz, Nuc. Acids Res. 12:3435-3439 (1984)). The indirect nature of the biotin labeling method limits the applicability for quantitative analysis, but the sensitivity of biotin assays are as high as that which can be achieved using radioisotopes.

The fluorescence detection systems require that excess label be washed off; furthermore, after hybridization real-time monitoring of the hybridization process is not feasible. In order to observe ongoing hybridization events on the surface, surface-related detection methods have been developed. These methods are based on different optical phenomenon on the surface and can detect subtle changes such as the formation of DNA duplexes on the surface, without interfering with the excess DNA in solution. Duplex electron transfer, optical wave-guide, surface plasmon resonance and resonant mirror are a few examples of currently developed surface-based detection methods (Wood, Microchem. J. 47:330-337 (1993); Stimpson and Gordon, Biomolecular Engineering 13:73-80 (1996); Wats et al., Biosensor. Anal. Chem. 67:4283-4289 (1995); and Stimpson et al., Proc. Natl. Acad. Sci. USA 92:6379-6380 (1995)). These methods have been tested for oligonucleotide array hybridization experiments utilizing a limited number of probes. The applicability of these methods to complex oligonucleotide arrays has yet to be evaluated.

### Embodiments of the Invention

### I. Arrays of HLA Oligonucleotide Probes

The present invention provides an array of HLA oligonucleotide probes. The array is a useful tool for performing phenotypic analysis on a tissue sample to determine, for example, whether a particular donor is suitable for matching in tissue transplantation. Generally, the array will comprise a series of oligonucleotide probes which represent at least 80%, preferably at least 90% and more preferably at least 98% of all known polymorphisms HLA Class I locus. In one preferred embodiment, the arrays will represent all known polymorphisms in exons 2 and 3 of the HLA Class I locus. The probes are provided on the array at known or preselected positions to facilitate analysis. Additionally, the probes are generally covalently attached to the solid support using a linking group that is sufficient to provide optimum binding of a sample nucleic acid to the probe array.

As used herein, the term “nucleic acid” or “oligonucleotide” refers to a deoxyribonucleotide or ribonucleotide in either single- or double-stranded form. The term also encompasses nucleic-acid-like structures with synthetic backbones. DNA backbone analogues provided by the invention include phosphodiester, phosphorothioate, phosphorodithioate, methylphosphonate, phosphoramidate, alkyl phosphotriester, sulfamate, 3′-thioacetal, methylene(methylimino), 3′-N-carbamate, morpholino carbamate, and peptide nucleic acids (PNAs); see Oligonucleotides and Analogues, a Practical Approach, edited by F. Eckstein, IRL Press at Oxford University Press (1991); Antisense Strategies, Annals of the New York Academy of Sciences, Volume 600, Eds. Baserga and Denhardt (NYAS 1992); Milligan (1993) J. Med. Chem. 36:1923-1937; Antisense Research and Applications (1993, CRC Press). PNAs contain non-ionic backbones, such as N-(2-aminoethyl) glycine units. Phosphorothioate linkages are described in WO 97/03211; WO 96/39154; Mata (1997) Toxicol. Appl. Pharmacol. 144:189-197. Other synthetic backbones encompasses by the term include methyl-phosphonate linkages or alternating methylphosphonate and phosphodiester linkages (Strauss-Soukup (1997) Biochemistry 36:8692-8698), and benzylphosphonate linkages (Samstag (1996) Antisense Nucleic Acid Drug Dev 6:153-156). The term nucleic acid is used interchangeably with gene, cDNA, mRNA, oligonucleotide primer, probe and amplification product.

The term “probe” or a “nucleic acid probe”, as used herein, is defined to be a collection of one or more nucleic acid fragments whose hybridization to a sample can be detected. The probe may be unlabeled or labeled as described below so that its binding to the target or sample can be detected. The probe is produced from a source of nucleic acids from one or more particular (preselected) portions of the genome, e.g., one or more clones, an isolated whole chromosome or chromosome fragment, or a collection of polymerase chain reaction (PCR) amplification products. Alternatively, the probes of the present invention are synthesized and have sequences corresponding to a source of nucleic acids. The probes of the present invention correspond to or are produced from nucleic acids found in the regions described herein. The probe or genomic nucleic acid sample may be processed in some manner, e.g., by removal of repetitive nucleic acids or enrichment with unique nucleic acids. The word “sample” may be used herein to refer not only to detected nucleic acids, but to the detectable nucleic acids in the form in which they are applied to the target. The probe may also be immobilized on a solid surface (e.g., nitrocellulose, glass, quartz, fused silica slides), as in an array. Techniques capable of producing high density arrays can also be used for this purpose (see, e.g., Fodor, supra (1991); Johnston, Curr. Biol. 8:R171-R174 (1998); Schummer, Biotechniques 23:1087-1092 (1997); Kern, Biotechniques 23:120-124 (1997); U.S. Pat. No. 5,143,854). One of skill will recognize that the precise sequence of the particular probes described herein can be modified to a certain degree to produce probes that are “substantially identical” to the disclosed probes, but retain the ability to specifically bind to (i.e., hybridize specifically to) the same targets or samples as the probe from which they were derived (see discussion above). Such modifications are specifically covered by reference to the individual probes described herein.

The term a “nucleic acid array” as used herein is a plurality of nucleic acid molecules (probes) immobilized on a solid surface (e.g., nitrocellulose, glass, quartz, fused silica slides and the like) to which sample nucleic acids are hybridized. The nucleic acids may contain sequence from specific genes or clones, such as the probes of the invention, as disclosed herein. Other probes optionally contain, for instance, reference sequences. The probes of the arrays may be arranged on the solid surface at different densities. The probe densities will depend upon a number of factors, such as the nature of the label, the solid support, and the like.

The array components are described in detail below.

Solid Supports

The solid support used in the present invention may be biological, nonbiological, organic, inorganic, or a combination of any of these, existing as particles, strands, precipitates, gels, sheets, tubing, spheres, containers, capillaries, pads, slices, films, plates, slides, etc. The solid support is preferably flat but may take on alternative surface configurations. For example, the solid support may contain raised or depressed regions on which synthesis takes place. In some embodiments, the solid support will be chosen to provide appropriate light-absorbing characteristics. For example, the support may be a polymerized Langmuir Blodgett film, functionalized glass, Si, Ge, GaAs, GaP, Sio₂, SiN₄, modified silicon, or any one of a variety of gels or polymers such as (poly)tetrafluoroethylene, (poly)vinylidendifluoride, polystyrene, polycarbonate, or combinations thereof. Other suitable solid support materials will be readily apparent to those of skill in the art. Preferably, the surface of the solid support will contain reactive groups, which could be carboxyl, amino, hydroxyl, thiol, or the like. More preferably, the surface will be optically transparent and will have surface Si—OH functionalities, such as are found on silica surfaces.

Linking Groups

Attached to the solid support is an optional spacer or linking group. The spacer molecules are preferably of sufficient length to permit the oligonucleotide probes in the completed array to interact freely with molecules exposed to the array. The spacer molecules, when present, are typically 6-50 atoms long to provide sufficient exposure for the attached probes. The spacer will typically be comprised of a surface attaching portion and a longer chain portion. The surface attaching portion is that part of the linking group or spacer which is directly attached to the solid support. This portion can be attached to the solid support via carbon-carbon bonds using, for example, supports having (poly)trifluorochloroethylene surfaces, or preferably, by siloxane bonds (using, for example, glass or silicon oxide as the solid support). Siloxane bonds with the surface of the support are formed in one embodiment via reactions of surface attaching portions bearing trichlorosilyl or trialkoxysilyl groups. The surface attaching groups will also have a site for attachment of the longer chain portion. For example, groups which are suitable for attachment to a longer chain portion would include amines, hydroxyl, thiol, and carboxyl. Preferred surface attaching portions include aminoalkylsilanes and hydroxyalkylsilanes. In particularly preferred embodiments, the surface attaching portion of the linking group is either aminopropyltriethoxysilane or aminopropyltrimethoxysilane.

The longer chain portion can be any of a variety of molecules which are inert to the subsequent conditions necessary for attaching the oligonucleotide probes, or for hybridization of a sample to the probe array. These longer chain portions will typically be ethylene glycol oligomers containing 2-14 monomer units, diamines, diacids, amino acids, peptides, or combinations thereof. In some embodiments, the longer chain portion is a polynucleotide (e.g., a 15-mer of poly dT; SEQ ID NO:141). Additionally, for use in synthesis of the probe arrays, the linking group will typically have a protecting group, attached to a functional group (i.e., hydroxyl, amino or carboxylic acid) on the distal or terminal end of the chain portion (opposite the solid support). After deprotection and coupling, the distal end is covalently bound to an oligonucleotide probe (e.g., an HLA Class I oligonucleotide probe).

HLA Oligonucleotide Probes

The key feature of the oligonucleotide array assay is the high redundancy of oligonucleotide probes. In one embodiment of the invention, oligonucleotide probes were designed to represent at least 80%, preferably at least 90% and more preferably at least 98% of the known polymorphisms in exon 2 and exon 3 of HLA-B. Known polymorphisms are those that have appeared in the literature or are available from a searchable database of sequences. A panel of 68 20-mer oligonucleotide probes were designed for polymorphisms in exon 2 and 70 20-mers were designed for exon 3. All known single allele in either homozygous samples or heterozygous samples could be distinguished from its hybridization pattern with this set of oligonucleotide probes, with the exception of three allele pairs.

The majority of individuals are heterozygous for two different HLA-B alleles. Sequence polymorphisms or “motifs” can be shared among families of HLA-B alleles at a given locus. Therefore, when both HLA-B alleles are co-amplified in PCR, more than one combination of two alleles may produce identical patterns of hybridization to oligonucleotide probes. Although alleles can be amplified separately with additional allele-specific PCR reactions, a multiplicity of amplification steps diminishes overall efficiency. The challenge of any molecular method is to identify all known coding region polymorphisms that would enable each allele to be unambiguously assigned. As used herein, the term “allele” refers to a specific version of a nucleotide sequence at a polymorphic genetic locus. A computer simulation using this oligonucleotide array has shown that all known single alleles in either homozygous samples or heterozygous samples could be distinguished from its hybridization pattern with this set of oligonucleotide probes, with the exception of five allele pairs mentioned above.

The fidelity of the hybridization assay is governed by the stability differences between perfectly matched and mismatched duplexes. Within one embodiment a single set of hybridization conditions that could provide a clear discrimination between matches and mismatches for all polymorphisms in each exon was determined. In order for the melting temperature of the probe sequences to be comparable, probes were designed with careful attention to probe size, base composition, and placement of mismatched position within the hybridization sequence.

Oligonucleotide probes of different lengths (15, 18 and 20 nucleotides) were tested to optimize the hybridization signal intensity and hybridization specificity. Probes were immobilized on solid supports and hybridized with PCR products. No signal was generated from 15-mer probes, and very weak signals were produced with 18-mer probes; the strongest signals were obtained with 20-mer probes. Thus, probes of between 17 and 23 nucleotides are useful within this invention. Within one embodiment, oligonucleotide probes of 20 nucleotides were used.

The length of the spacer between the support and the hybridization sequence influences the efficiency of hybridization (Guo et al, Nuc. Acids Res. 22:5456-5465 (1994)). When large DNA fragments, such as PCR products, are allowed to hybridize with short oligonucleotide probes immobilized on solid supports, adequate distance between the hybridization sequence and the solid surface is required in order to achieve the efficient hybridization. This is due to the steric interference between large DNA molecules and the support. Within one embodiment of the invention, a 15-mer dT spacer (SEQ ID NO:141) was employed in each oligonucleotide probe to provide adequate space between hybridization sequence and the support. Although requiring extra expense in oligonucleotide synthesis, the 15-mer spacer was essential to optimize hybridization signals. Each completed probe contained a 5′ amino group for immobilization chemistry, a 20-nucleotide hybridization sequence, and a 15-mer dT spacer (SEQ ID NO:141) between them.

As noted above, the oligonucleotide probes useful in this aspect of the invention are those probes that represent at least 80%, preferably 90%, more preferably 98% and most preferably all the known polymorphisms in exons 2 and 3 of the HLA Class I locus. The probes will generally comprise from about 17 to 23 nucleic acid residues (excluding a linking oligonucleotide) with those having about 20 nucleic acid residues being preferred.

In a preferred embodiment, the HLA Class I oligonucleotide probes are HLA-A oligonucleotide probes, HLA-B oligonucleotide probes or HLA-C oligonulceotide probes. In one group of particularly preferred embodiments, the oligonucleotides probes are those HLA-B probes provided in Tables 1 and 2.

TABLE 1

| | | | |
|---|---|---|---|
| | | | Seq. |
| Probe | Location | Sequence | I.D. No. |

PCR Probe HLA Exon Location 5-24

| | | | |
|---|---|---|---|
| A1 | 05-24 | 5′TCACACCCTCCAGAGCATGT3′ | 1 |
| A2 | 05-24 | 5′TCACATCATCCAGAGGATGT3′ | 2 |
| A3 | 05-24 | 5′TCACACTTGGCAGACGATGT3′ | 3 |
| A4 | 05-24 | 5′TCACACCCTCCAGTGGATGT3′ | 4 |
| A5 | 05-24 | 5′TCACACTTGGCAGAGGATGT3′ | 5 |
| A6 | 05-24 | 5′TCACACCCTCCAGACGATGT3′ | 6 |
| A7 | 05-24 | 5′TCACACCCTCCAGAATATGT3′ | 7 |
| A8 | 05-24 | 5′TCACATCATCCAGAGCATGT3′ | 8 |
| A9 | 05-24 | 5′TCACACCATCCAGAGGATGT3′ | 9 |
| A10 | 05-24 | 5′TCACATCATCCAGGTGATGT3′ | 10 |
| A11 | 05-24 | 5′TCACACCCTCCAGAGGATGT3′ | 11 |

PCR Probe HLA Exon Location 21-40

| | | | |
|---|---|---|---|
| B1 | 21-40 | 5′ATGTACGGCTGCGACGTGGG3′ | 12 |
| B2 | 21-40 | 5′ATGTATGGCTGCGACCTGGG3′ | 13 |
| B3 | 21-40 | 5′ATGTTTGGCTGCGACGTGGG3′ | 14 |
| B4 | 21-40 | 5′ATGTAAGGCTGCGACGTGGG3′ | 15 |
| B5 | 21-40 | 5′ATGTCTGGCTGCGACGTGGG3′ | 16 |
| B6 | 21-40 | 5′ATGTACGGCTGCGACCTGGG3′ | 17 |
| B7 | 21-40 | 5′ATGTTTGGCTGCGACCTGGG3′ | 18 |
| B8 | 21-40 | 5′ATGTATGGCTGCGACATGGG3′ | 19 |
| B9 | 21-40 | 5′ATGTATGGCTGCGACGTGGG3′ | 20 |

PCR Probe HLA Exon Location 41-60

| | | | |
|---|---|---|---|
| C1 | 41-60 | 5′GCCCGACGGGCGCCTCCTCC3′ | 21 |
| C2 | 41-60 | 5′GCCCGACGGGCGCTTCCTCC3′ | 22 |
| C3 | 41-60 | 5′GCCGGACGGGCGCCTCCTCC3′ | 23 |

PCR Probe HLA Exon Location 61-80

| | | | |
|---|---|---|---|
| D1 | 61-80 | 5′GCGGGCATGACCAGTACGCC3′ | 24 |
| D2 | 61-80 | 5′GCGGGCATAACCAGTACGCC3′ | 25 |
| D3 | 61-80 | 5′GCGGGCATAACCAGTTAGCC3′ | 26 |
| D4 | 61-80 | 5′GCGGGTATAACCAGTTCGCC3′ | 27 |
| D5 | 61-80 | 5′GCGGGCATGACCAGTCCGCC3′ | 28 |
| D6 | 61-80 | 5′GCGGGTATGACCAGTCCGCC3′ | 29 |
| D7 | 61-80 | 5′GCGGGTACCACCAGGACGCC3′ | 30 |
| D8 | 61-80 | 5′GCGGGCATGACCAGTTCGCC3′ | 31 |
| D9 | 61-80 | 5′GCGGGCATAACCAGTTCGCC3′ | 32 |
| D10 | 61-80 | 5′GCGGGTATGACCAGGACGCC3′ | 33 |
| D11 | 61-80 | 5′GCGGGTATAACCAGTTAGCC3′ | 34 |
| D12 | 61-80 | 5′GCGGGTATGACCAGTACGCC3′ | 35 |

PCR Probe HLA Exon Location 81-100

| | | | |
|---|---|---|---|
| E1 | 81-100 | 5′TACGACGGCAAAGATTACAT3′ | 36 |
| E2 | 81-100 | 5′TACGACGGCAAGGATTACAT3′ | 37 |

PCR Probe HLA Exon Location 111-130

| | | | |
|---|---|---|---|
| F1 | 111-130 | 5′GAGGACCTGAGCTCCTGGAC3′ | 38 |
| F2 | 111-130 | 5′GAGGACCTGCGCTCCTGGAC3′ | 39 |

PCR Probe HLA Exon Location 131-150

| | | | |
|---|---|---|---|
| G1 | 131-150 | 5′CGCCGCGGACACGGCGGCTC3′ | 40 |
| G2 | 131-150 | 5′CGCGGCGGACACCGCGGCTC3′ | 41 |
| G3 | 131-150 | 5′CGCCGCGGACAAGGCGGCTC3′ | 42 |
| G4 | 131-150 | 5′CGCCGCGGACACGGCAGCTC3′ | 43 |
| G5 | 131-150 | 5′CGCCGCGGACACCGCGGCTC3′ | 44 |
| G6 | 131-150 | 5′CGCGGCGGACACGGCGGCTC3′ | 45 |

PCR Probe HLA Exon Location 151-170

| | | | |
|---|---|---|---|
| H1 | 151-170 | 5′AGATCACCCAGCTCAAGTGG3′ | 46 |
| H2 | 151-170 | 5′AGATCTCCCAGCGCAAGTTG3′ | 47 |
| H3 | 151-170 | 5′AGATCACCCAGCGCAAGTGG3′ | 48 |

PCR Probe HLA Exon Location 171-190

| | | | |
|---|---|---|---|
| I1 | 171-190 | 5′GAGGCGGCCCGTGAGGCGGA3′ | 49 |
| I2 | 171-190 | 5′GAGGCGGCCCGTGTGGCGGA3′ | 50 |

PCR Probe HLA Exon Location 191-210

| | | | |
|---|---|---|---|
| J1 | 191-210 | 5′GCAGCGGAGAGCCTACCTGG3′ | 51 |
| J2 | 191-210 | 5′GCAGGACAGAGCCTACCTGG3′ | 52 |
| J3 | 191-210 | 5′GCAGTGGAGAGCCTACCTGG3′ | 53 |
| J4 | 191-210 | 5′GCAGCTGAGAACCTACCTGG3′ | 54 |
| J5 | 191-210 | 5′GCAGCGGAGAACCTACCTGG3′ | 55 |
| J6 | 191-210 | 5′GCAGCTGAGAGCCTACCTGG3′ | 56 |

PCR Probe HLA Exon Location 211-230

| | | | |
|---|---|---|---|
| K1 | 211-230 | 5′AGGGCGAGTGCGTGGAGTGG3′ | 57 |
| K2 | 211-230 | 5′AGGGCCTGTGCGTGGAGTGG3′ | 58 |
| K3 | 211-230 | 5′AGGGCCTGTGCGTGGACGGG3′ | 59 |
| K4 | 211-230 | 5′AGGGCCTGTGCGTGGAGTCG3′ | 60 |
| K5 | 211-230 | 5′AGGGCACGTGCGTGGAGTCG3′ | 61 |
| K6 | 211-230 | 5′AGGGCCTGTGCGTGGAGGGG3′ | 62 |
| K7 | 211-230 | 5′AGGACCTGTGCGTGGAGTCG3′ | 63 |
| K8 | 211-230 | 5′AGGGCACGTGCGTGGAGTGG3′ | 64 |

PCR Probe HLA Exon Location 231-250

| | | | |
|---|---|---|---|
| L1 | 231-250 | 5′CTCCGCAGACACCTGGAGAA3′ | 65 |
| L2 | 231-250 | 5′CTCCGCAGATACCTGGAGAA3′ | 66 |

PCR Probe HLA Exon Location 257-276

| | | | |
|---|---|---|---|
| M1 | 257-276 | 5′GGACAAGCTGGAGCGCGCTG3′ | 67 |
| M2 | 257-276 | 5′GGACACGCTGGAGCGCGCGG3′ | 68 |
| M3 | 257-276 | 5′GGAGACGCTGCAGCGCGCGG3′ | 69 |

The oligonucleotide probes used in the present invention can be prepared by any of a variety of methods. Briefly, the probes can be prepared using i) solution or solid phase methods, followed by attachment to the solid support, or ii) solid phase methods wherein the probes are constructed on the array surface.

Solution or Solid Phase Methods

Detailed descriptions of the procedures for solution and solid phase synthesis of nucleic acids by phosphite-triester, phosphotriester, and H-phosphonate chemistries are widely available. For example, the solid phase phosphoramidite triester method of Beaucage and Carruthers using an automated synthesizer is described in, e.g., Itakura, U.S. Pat. No. 4,401,796; Carruthers, U.S. Pat. Nos. 4,458,066 and 4,500,707. See also Needham-VanDevanter, Nucleic Acids Res. 12:6159-6168 (1984); Beigelman Nucleic Acids Res 23:3989-3994 (1995); OLIGONUCLEOTIDE SYNTHESIS: A PRACTICAL APPROACH, Gait (ed.), IRL Press, Washington D.C. (1984), see Jones, chapt 2, Atkinson, chapt 3, and Sproat, chapt 4; Froehler, Tetrahedron Lett. 27:469-472 (1986); Froehler, Nucleic Acids Res. 14:5399-5407 (1986). Methods to purify oligonucleotides include native acrylamide gel electrophoresis, anion-exchange HPLC, as described in Pearson J. Chrom. 255:137-149 (1983). The sequence of the synthetic oligonucleotide can be verified using any chemical degradation method, e.g., see Maxam (1980) Methods in Enzymology 65:499-560, Xiao Antisense Nucleic Acid Drug Dev 6:247-258 (1996), or for solid-phase chemical degradation procedures, Rosenthal, Nucleic Acids Symp. Ser. 18:249-252 (1987).

Solid-support Based Oligonucleotide Synthesis

An array of oligonucleotide probes at known locations on a single substrate surface can be formed using a variety of techniques known to those skilled in the art of polymer synthesis on solid supports. For example, “light directed” methods (which are one technique in a family of methods known as VLSIPS™ methods) are described in U.S. Pat. No. 5,143,854, previously incorporated by reference. The light directed methods discussed in the '854 patent involve activating predefined regions of a substrate or solid support and then contacting the substrate with a preselected monomer solution. The predefined regions can be activated with a light source shown through a mask (much in the manner of photolithography techniques used in integrated circuit fabrication). Other regions of the substrate remain inactive because they are blocked by the mask from illumination and remain chemically protected. Thus, a light pattern defines which regions of the substrate react with a given monomer. By repeatedly activating different sets of predefined regions and contacting different monomer solutions with the substrate, a diverse array of polymers is produced on the substrate. Of course, other steps such as washing unreacted monomer solution from the substrate can be used as necessary.

Other useful techniques include mechanical techniques (e.g., flow channel, spotting or pin-based methods). In each of the “flow channel” or “spotting” methods, certain activated regions of the substrate are mechanically separated from other regions when the monomer solutions are delivered to the various reaction sites.

A typical “flow channel” method applied to the compounds and libraries of the present invention can generally be described as follows. Diverse probe sequences are synthesized at selected regions of a substrate or solid support by forming flow channels on a surface of the substrate through which appropriate reagents flow or in which appropriate reagents are placed. For example, assume a monomer “A” is to be bound to the substrate in a first group of selected regions. If necessary, all or part of the surface of the substrate in all or a part of the selected regions is activated for binding by, for example, flowing appropriate reagents through all or some of the channels, or by washing the entire substrate with appropriate reagents. After placement of a channel block on the surface of the substrate, a reagent having the monomer A flows through or is placed in all or some of the channel(s). The channels provide fluid contact to the first selected regions, thereby binding the monomer A on the substrate directly or indirectly (via a spacer) in the first selected regions.

Thereafter, a monomer B is coupled to second selected regions, some of which may be included among the first selected regions. The second selected regions will be in fluid contact with a second flow channel(s) through translation, rotation, or replacement of the channel block on the surface of the substrate; through opening or closing a selected valve; or through deposition of a layer of chemical or photoresist. If necessary, a step is performed for activating at least the second regions. Thereafter, the monomer B is flowed through or placed in the second flow channel(s), binding monomer B at the second selected locations. In this particular example, the resulting sequences bound to the substrate at this stage of processing will be, for example, A, B, and AB. The process is repeated to form an array of sequences of desired length at known locations on the substrate.

After the substrate is activated, monomer A can be flowed through some of the channels, monomer B can be flowed through other channels, a monomer C can be flowed through still other channels, etc. In this manner, many or all of the reaction regions are reacted with a monomer before the channel block must be moved or the substrate must be washed and/or reactivated. By making use of many or all of the available reaction regions simultaneously, the number of washing and activation steps can be minimized.

One of skill in the art will recognize that there are alternative methods of forming channels or otherwise protecting a portion of the surface of the substrate. For example, according to some embodiments, a protective coating such as a hydrophilic or hydrophobic coating (depending upon the nature of the solvent) is utilized over portions of the substrate to be protected, sometimes in combination with materials that facilitate wetting by the reactant solution in other regions. In this manner, the flowing solutions are further prevented from passing outside of their designated flow paths.

The “spotting” methods of preparing compounds and libraries of the present invention can be implemented in much the same manner as the flow channel methods. For example, a monomer A can be delivered to and coupled with a first group of reaction regions which have been appropriately activated. Thereafter, a monomer B can be delivered to and reacted with a second group of activated reaction regions. Unlike the flow channel embodiments described above, reactants are delivered by directly depositing (rather than flowing) relatively small quantities of them in selected regions. In some steps, of course, the entire substrate surface can be sprayed or otherwise coated with a solution. In preferred embodiments, a dispenser moves from region to region, depositing only as much monomer as necessary at each stop. Typical dispensers include a micropipette to deliver the monomer solution to the substrate and a robotic system to control the position of the micropipette with respect to the substrate. In other embodiments, the dispenser includes a series of tubes, a manifold, an array of pipettes, or the like so that various reagents can be delivered to the reaction regions simultaneously.

Another method which is useful for the preparation of an array of diverse oligonucleotides on a single substrate involves “pin based synthesis.” This method is described in detail in U.S. Pat. No. 5,288,514, previously incorporated herein by reference. The method utilizes a substrate having a plurality of pins or other extensions. The pins are each inserted simultaneously into individual reagent containers in a tray. In a common embodiment, an array of 96 pins/containers is utilized.

Each tray is filled with a particular reagent for coupling in a particular chemical reaction on an individual pin. Accordingly, the trays will often contain different reagents. Since the chemistry used is such that relatively similar reaction conditions may be utilized to perform each of the reactions, multiple chemical coupling steps can be conducted simultaneously. In the first step of the process, a substrate on which the chemical coupling steps are conducted is provided. The substrate is optionally provided with a spacer (e.g., 15-mer of poly-dT) having active sites on which the oligonucleotide probes are attached or constructed.

### II. Methods of Preparing Oligonucleotide Probe Arrays

In another aspect, the present invention provides methods of preparing oligonucleotide probe arrays. In this group of embodiments, oligonucleotide probe arrays are prepared by:
(a) contacting a solid support with an aminoalkyltrialkoxysilane in the vapor phase at reduced pressure to form an aminoalkylsilane-derivatized solid support;
(b) contacting the aminoalkylsilane-derivatized solid support with a linking group to covalently attach the linking group to the aminoalkylsilane-derivatized solid support to form a linking group-modified solid support; and
(c) attaching a plurality of oligonucleotide probes to the linking group-modified solid support to form the array of covalently-attached oligonucleotide probes.

The solid supports used in this aspect of the invention can be any of those described above which are conveniently derivatized with a vapor phase deposition of an aminoalkyltrialkoxysilane. Surprisingly, the use of this vapor phase deposition technique provides a particularly uniform surface for probe assembly and presentation.

The aminoalkyltrialkoxysilanes useful in this aspect of the invention are any of those that can be utilized in the vapor phase at temperatures of from about ambient temperature to about 150° C. at pressures of from about 760 mmHg to about 0.1 mmHg. Typically, the aminoalkyl portion of the silane will be aminopropyl, aminoethyl or aminomethyl. The trialkoxysilane portion can be one in which the alkoxy groups are all the same (e.g., trimethoxysilane, triethoxysilane) or one in which the alkoxy groups are not all alike (e.g., dimethoxyethoxysilane). Accordingly, the aminoalkyltrialkoxysilane will typically be selected from aminopropyltrimethoxysilane, aminopropyltriethoxysilane, aminopropyldiethoxymethoxysilane, aminoethyltrimethoxysilane, and the like. More preferably, the aminoalkyltrialkoxysilane is aminopropyltrimethoxysilane.

As indicated above, a more uniform coating of amino groups on the solid support can be achieved by applying an aminoalkyltrialkoxysilane in the vapor phase, typically at reduced pressure. This can be accomplished by placing the solid support into a vacuum chamber, evacuating the chamber, and introducing the silane. In some embodiments, the vacuum chamber can be heated to facilitate silane vaporization and even coating of the solid support. For example, when aminopropyltrimethoxysilane is used, the pressure will typically be from about 5 to 35 mmHg and the vacuum chamber will be heated to a temperature of from about 60 to about 110° C. After a period of time sufficient for formation of an aminoalkylsilane-derivatized solid support, the support is removed from the vacuum chamber and rinsed to remove any unbound spacer.

The resultant support can then be contacted with a suitable amount of a linking group to covalently attach the linking group to the aminoalkylsilane-derivatized solid support. In some embodiments, the aminoalkylsilane-derivatized solid support will first be treated with a reagent capable of facilitating linking group attachment to the derivatized support. A variety of reagents are useful in this aspect of the invention including diisocyanates, diisothiocyanates, dicarboxylic acids (and their activated esters), and the like. Particular preferred are diisothiocyanates (e.g. 1,4-phenylenediisothiocyanate).

Once the solid support has been suitably derivatized, a linking group is attached to provide a spacing between the oligonucleotide probe and the support which is optimized for interactions between the probes and the sample. As provided above, a variety of linking groups can be used in this aspect of the invention. Preferred groups are those that provide a spacing similar to that provided by a 15-mer poly dT spacing group (SEQ ID NO:141). Additionally, the linking group will have a reactive portion that is selected to be compatible with the amino group of the aminoalkylsilane-derivatized support, or with the functional group present on the reagent used to facilitate linking group attachment (e.g., the isothiocyanate portion of 1,4-phenylenediisothiocyanate). Accordingly, at the proximal end (that forming an attachment closest to the support), the linking group will have a functional group that is reactive with an amino moiety (e.g., a carboxylic acid, anhydride, isothiocyanate, and the like) or a functional group that is reactive with an isocyanate, isothiocyanate or carboxylic acid moiety (e.g., an amino group, a hydroxyl group or the like).

In a particularly preferred embodiment, the support is derivatized first with aminopropyltrimethoxysilane, followed by attachment of 1,4-phenylenediisothiocyanate, followed by attachment of a 15-mer oligonucleotide, preferably a 15-mer of poly-dT (SEQ ID NO:141).

Following construction of the linking group-modified solid support, a plurality of oligonucleotide probes is attached to form an array of covalently-attached oligonucleotide probes. In this aspect of the invention, the oligonucleotide probes can be any collection of nucleic acid probes or polymer. Preferably, the probes are those that represent all known polymorphisms in exons 2 and 3 of the HLA Class I locus. The probes are typically 17 to 23 nucleotides in length, with those probes having about 20 nucleotides being particularly preferred. The most preferred HLA Class I oligonucleotide probes are shown in Tables 1 and 2. The oligonucleotide probes can be prepared by any conventional methods known to those of skill in the art. Alternatively, the probes can be constructed on the array using the techniques described above (e.g., photolithography, flow channel, ink-jet spotting, and the like). In preferred embodiments, the probes are construct using convention solution or solid phase chemistry, then attached to the array's solid support component.

Construction of the present arrays is preferably carried out in a manner that ensures that the probes are at a surface density of about 250 to about 450 angstrom²/molecule, preferably about 325 to about 375 angstrom²/molecule. Methods of measuring probe density are well-known to those of skill in the art.

III. Diagnostic Methods Using Oligonucleotide Arrays

In order to determine donor/recipient compatibility in tissue transplants, the practitioner should compare the HLA Class I allele type of both the donor and the recipient. Tools to facilitate such tissue typing are provided herein.

Accordingly, in still another aspect, the present invention provides a method of HLA Class I tissue typing, the method comprising:
(a) amplifying exons 2 and 3 from a genomic sample of tissue using labeled primers and an asymmetric PCR method to form a labeled, single-stranded DNA sample;
(b) contacting the labeled, single-stranded DNA sample under hybridization conditions with an array of HLA Class I oligonucleotide probes prepared by the methods described herein; and
(c) detecting a hybridization pattern for the DNA sample and assigning an HLA Class I allele type by analysis of the hybridization pattern.

In this method, a tissue sample is obtained from a patient (either a potential donor or recipient) and exons 2 and 3 are amplified using labeled primers and an asymmetric PCR method to form a labeled, single-stranded DNA sample. The tissue sample can be obtained from a variety of tissues, depending on the purpose of the diagnostic evaluation. The cell or tissue sample from which the nucleic acid sample is prepared is typically taken from a patient in need of HLA Class I tissue typing for transplant evaluation. Methods of isolating cell and tissue samples are well known to those of skill in the art and include, but are not limited to, aspirations, tissue sections, needle biopsies, and the like. Frequently the sample will be a “clinical sample” which is a sample derived from a patient, including sections of tissues such as frozen sections or paraffin sections taken for histological purposes. The sample can also be derived from supernatants (of cells) or the cells themselves from cell cultures, cells from tissue culture and other media.

After obtaining a suitable tissue sample, the nucleic acids of exons 2 and 3 are amplified using standard techniques such as PCR (e.g., asymmetric PCR) and labeled primers. The term “labeled primer” as used herein refers to a nucleic acid template for PCR which is attached to a detectable composition, i.e., a label. The detection of the label can be by, e.g., spectroscopic, photochemical, biochemical, immunochemical, physical or chemical means. For example, useful labels include ³²P, ³⁵S, ³H, ¹⁴C, ¹²⁵I, ¹³¹I; fluorescent dyes (e.g., FITC, rhodamine, lanthanide phosphors, Texas red), electron-dense reagents (e.g. gold), enzymes, e.g., as commonly used in an ELISA (e.g., horseradish peroxidase, beta-galactosidase, luciferase, alkaline phosphatase), colorimetric labels (e.g. colloidal gold), magnetic labels (e.g. Dynabeads™), biotin, dioxigenin, or haptens and proteins for which antisera or monoclonal antibodies are available. The label can be directly incorporated into the nucleic acid to be detected. Additionally, the label can be attached by spacer arms of various lengths to reduce potential steric hindrance or impact on other useful or desired properties. See, e.g., Mansfield, Mol Cell Probes 9:145-156 (1995).

The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions. The term “stringent conditions” or “hybridization conditions” refers to conditions under which a probe will hybridize preferentially to its target subsequence, and to a lesser extent to, or not at all to, other sequences. A “stringent hybridization” and “stringent hybridization wash conditions” in the context of nucleic acid hybridization (e.g., as in array, Southern or Northern hybridizations) are sequence dependent, and are different under different environmental parameters. An extensive guide to the hybridization of nucleic acids is found in, e.g., Tijssen (1993) Laboratory Techniques in Biochemistry and Molecular Biology—Hybridization with Nucleic Acid Probes part I, chapt 2, “Overview of principles of hybridization and the strategy of nucleic acid probe assays,” Elsevier, N.Y. (“Tijssen”). Generally, highly stringent hybridization and wash conditions are selected to be about 5° C. lower than the thermal melting point (Tₘ) for the specific sequence at a defined ionic strength and pH. The Tm is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on an array or on a filter in a Southern or northern blot is 42° C. using standard hybridization solutions (see, e.g., Sambrook and detailed discussion, below), with the hybridization being carried out overnight. An example of highly stringent wash conditions is 0.15 M NaCl at 72° C. for about 15 minutes. An example of stringent wash conditions is a 0.2×SSC wash at 65° C. for 15 minutes (see, e.g., Sambrook (1989) Molecular Cloning: A Laboratory Manual (2nd ed.) Vol. 1-3, Cold Spring Harbor Laboratory, Cold Spring Harbor Press, N.Y. (“Sambrook”) for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example of a low stringency wash for a duplex of, e.g., more than 100 nucleotides, is 4× to 6×SSC at 40° C. for 15 minutes. Within one embodiment of the invention, hybridization at 37° C. for two hours in 5×SSPE, 0.5% SDS was followed by two fifteen minute washes at stringent conditions in 20×SSPE, 0.2% SDS at 30° C.

In an array format a large number of different hybridization reactions can be run essentially “in parallel.” This provides rapid, essentially simultaneous, evaluation of a large number of loci. Methods of performing hybridization reactions in array based formats are also described in, e.g., Pastinen (1997) Genome Res. 7:606-614; (1997) Jackson (1996) Nature Biotechnology 14:1685; Chee (1995) Science 274:610; WO 96/17958.

To optimize a given assay format, one of skill can determine sensitivity of label (e.g., fluorescence) detection for different combinations of membrane type, fluorochrome, excitation and emission bands, spot size and the like. Low fluorescence background membranes can be used (see, e.g., Chu (1992) Electrophoresis 13:105-114). The sensitivity for detection of spots (“target elements”) of various diameters on the candidate membranes can be readily determined by, e.g., spotting a dilution series of fluorescently end labeled DNA fragments. These spots are then imaged using conventional fluorescence microscopy. The sensitivity, linearity, and dynamic range achievable from the various combinations of fluorochrome and solid surfaces (e.g., membranes, glass, fused silica) can thus be determined. Serial dilutions of pairs of fluorochrome in known relative proportions can also be analyzed. This determines the accuracy with which fluorescence ratio measurements reflect actual fluorochrome ratios over the dynamic range permitted by the detectors and fluorescence of the substrate upon which the probe has been fixed.

Arrays on solid surface substrates with much lower fluorescence than membranes, such as glass, quartz, or small beads, can achieve much better sensitivity. Substrates such as glass or fused silica are advantageous in that they provide a very low fluorescence substrate, and a highly efficient hybridization environment. Covalent attachment of the target nucleic acids to glass or synthetic fused silica can be accomplished according to a number of known techniques (described above). Nucleic acids can be conveniently coupled to glass using commercially available reagents. For instance, materials for preparation of silanized glass with a number of functional groups are commercially available or can be prepared using standard techniques (see, e.g., Gait (1984) Oligonucleotide Synthesis: A Practical Approach, IRL Press, Wash., D.C.). Quartz cover slips, which have at least 10-fold lower autofluorescence than glass, can also be silanized.

Alternatively, probes can also be immobilized on commercially available coated beads or other surfaces. For instance, biotin end-labeled nucleic acids can be bound to commercially available avidin-coated beads. Streptavidin or anti-digoxigenin antibody can also be attached to silanized glass slides by protein-mediated coupling using e.g., protein A following standard protocols (see, e.g., Smith (1992) Science 258:1122-1126). Biotin or digoxigenin end-labeled nucleic acids can be prepared according to standard techniques. Hybridization to nucleic acids attached to beads is accomplished by suspending them in the hybridization mix, and then depositing them on the glass substrate for analysis after washing. Alternatively, paramagnetic particles, such as ferric oxide particles, with or without avidin coating, can be used.

In one particularly preferred embodiment, probe nucleic acid is spotted onto a surface (e.g., a glass or quartz surface). The nucleic acid is dissolved in a mixture of dimethylsulfoxide (DMSO) and nitrocellulose and spotted onto amino-silane coated glass slides. Small capillaries tubes can be used to “spot” the probe mixture.

In related aspects, the present invention provides a method of HLA tissue typing, the method comprising:
(a) selectively amplifying the HLA regions in a genomic sample using asymmetric PCR and labeled primers to form a labeled, single-stranded DNA sample;
(b) contacting under hybridization conditions the labeled, single-stranded DNA sample with an HLA microarray prepared by any of the methods described herein; and
(c) detecting a hybridization pattern for the DNA sample and assigning an HLA allele type by analysis of the hybridization pattern.

In another related aspect, the present invention provides a method of HLA-B tissue typing, the method comprising:
(a) amplifying exons 2 and 3 from a genomic sample of tissue using labeled primers and an asymmetric PCR method to form a labeled, single-stranded DNA sample;
(b) contacting under hybridization conditions, the labeled, single-stranded DNA sample with any or the HLA-B microarrays described herein; and
(c) detecting a hybridization pattern for the DNA sample and assigning an HLA-B allele type by analysis of the hybridization pattern.

### EXAMPLES

The following examples are offered to illustrate, but not to limit the scope of the claimed invention.

### EXAMPLE 1

This example illustrates the constructions of HLA-B oligonucleotide probes used in constructing the probe arrays.

The key feature of the oligonucleotide array assay is the high redundancy of oligonucleotide probes. Oligonucleotide probes were designed to represent all known polymorphisms in exon 2 and exon 3 of HLA-B. A panel of 68 20-mer oligonucleotide probes were designed for polymorphisms in exon 2 (Table 1) and 70 20-mers were designed for exon 3 (Table 2). All known single allele in either homozygous samples or heterozygous samples could be distinguished from its hybridization pattern with this set of oligonucleotide probes, with the exception of three allele pairs. All oligonucleotides were synthesized by Life Technologies, Inc. (Frederick, Md.). The oligonucleotide probes used in manufacture of oligonucleotide arrays containing a 5′-amino group for immobilization chemistry. Concentrations of all oligonucleotides were determined by UV spectrophotometry at 260 nm. Oligonucleotide probes of different lengths (15, 18 and 20 nucleotides) were tested to optimize the hybridization signal intensity and hybridization specificity. Probes were immobilized on solid supports and hybridized with PCR products. No signal was generated from 15-mer probes, and very weak signals were produced with 18-mer probes; the strongest signals were obtained with 20-mer probes. Therefore, a hybridization sequence of 20 nucleotides in length was chosen for all HLA-B oligonucleotides.

### EXAMPLE 2

This example illustrates the construction of HLA-B oligonucleotide arrays.

HLA-B oligonucleotide arrays were constructed on treated microscopic slides by attaching pre-synthesized oligonucleotide probes. The arrays for exon 2 and exon 3 were fabricated on separate slides. Oligonucleotide probes were diluted to 500 pmole/mL, transferred into 96-well microtiter plate, applied to glass slides by using a Molecular Dynamic (Sunnyvale, Calif.) spotter system and immobilized on glass supports by covalent binding. Pre-cleaned microscope slides (Becton, Dickinson and Co., Portsmouth, N.H.) were immersed in concentrated HCl for 2 hours, then washed ten times with distilled water, five minutes per wash, and air dried. The cleaned slides were placed in a vacuum chamber with 700 microliters 3-aminopropyltimethoxysilane (Aldrich Chemical, Milwaukee, Wis.) and the vacuum chamber was kept at 160° C. and 30 ppm Hg pressure for 3 hours. The slides were taken from the vacuum chamber and washed 5 times with acetone, five minutes per wash, and then treated for 2 hours with a solution of 0.2% 1,4-phenylene diisothiocyanate (Aldrich) in 10% pyridine/dimethyl formamide, and washed with acetone for 5 times, 5 minutes per wash. The activated glass slides may be stored indefinitely at 4° C. in a vacuum dessicator containing anhydrous calcium chloride without discernible loss of activity. Oligonucleotide probes, labeled with 5′-amino group, were dissolved at concentration of 500 pm/ul in water. Twenty-five microliters of the resultant solutions were transferred into 96-well microtiter plates. The filled microtiter plates were then placed into a Molecular Dynamic Generation II array spotter, along with the activated glass slides. The spotter system was designed to automatically collect samples from a 96-well microtiter plate with a 6-pen robot arm. Each pen collected from between 250 to 500 nL of solution per pen and deposited 0.25-1 nL on each slide, creating spots that ranged from 100-150 micron in diameter. The robot was programmed so that adjacent spots were spaced to avoid contact with each other, with 400-500 microns separating the centers of each spot. The precision of this measurement is about 10 microns. The robot rested on an optical table where 24 glass slides could be placed. At maximal capacity, 3,000 different oligonucleotides could be arrayed on one glass slide. The transfer of oligonucleotide solution from microtiter plate to glass slides was conducted at 50% humidity, and each oligonucleotide solution were spotted at four different places near each other on the glass slides. The spotting step was repeated three times for each glass slide. Slides spotted with oligonucleotide probes were then incubated at 37° C. in a covered petri dish containing a small amount of water for 2 hours, removed, washed once with 1% NH₄OH, three times with water, and air-dried at room temperature. The slides were now ready for hybridization experiments. It is not recommended that the slides be employed multiple times, as rapidly increased background is observed.

The oligonucleotide probes were linked to the glass surface by covalent bonding (Guo et al., Nuc. Acids Res. 22:5456-5465 (1994)). The immobilization chemistry included three steps: a) reaction of the pre-cleaned glass slides with aminopropyltrimethoxylsilane vapor in vacuum chamber to generate an amino-derivatized surface; b) coupling of the amino group on the glass surface with excess p-phenylenediisothiocyanate to convert the amino groups to amino-reactive phenylisothiocyanate groups; and c) coupling of 5′-amino modified oligonucleotide probes to these amino-reactive groups to yield the surface-bound oligonucleotide.

Efficient and stable oligonucleotide coupling was achieved using this immobilization chemistry. Oligonucleotide arrays could be washed with water and stored at room temperature for a considerable period without any observable loss of oligonucleotides. The surface density of each oligonucleotide probe could be easily adjusted by changing the concentration of the oligonucleotide solution during the application step period.

### EXAMPLE 3

This example illustrates the preparation of DNA samples.

Human genomic DNA samples encoding various HLA-B genotypes were studied. The OD 260/280 measurements of the DNA samples were used to assess the quality of genomic DNA. Exon 2 and exon 3 of HLA-B were amplified into fragments of 270 bp and 276 bp, respectively, using HLA-B specific primers, under optimized conditions (Petersdorf and Hansen, Tissue Antigen 46: 73-85 (1995)). One primer out of each primer pair was tagged with a 6-Rhodamine dye moiety (ABI) at its 5′-end for fluorescence detection after hybridization. The PCR products were purified by reverse-phase high performance liquid chromatography. The specificity of the amplified product was verified by conventional sequencing methods.

Although it is simpler to prepare double-stranded PCR products than single-stranded, hybridization of the double-stranded DNA to the support-bound oligonucleotide array will necessarily suffer from competition of the complementary strand with the oligonucleotide probes. Hybridization efficiency is much greater with the single-stranded HLA-B PCR product compared to the double-stranded PCR products (Guo et al., Nuc. Acids Res. 22: 5456-5465 (1994)). Accordingly, several methods were explored to generate single-stranded PCR products, including strand separation using biotin-streptavidin interaction, 1 exonuclease digest and asymmetric PCR. Separation of double-stranded DNA was accomplished by labeling one PCR primer with a biotin molecule at its 5′-end; streptavidin-coated magnetic beads were added after PCR amplification; the biotinylated PCR product was attached to the beads by biotin-streptavidin interaction. The two DNA strands were separated through NaOH precipitation of the biotinylated strand using a magnet (Guo et al., supra. (1994)).

The exonuclease digest approach utilized 1 exonuclease, which digests double-stranded DNA molecules from its 5′-end while leaving single-stranded DNA molecules intact. HLA-B was amplified from genomic DNA using one fluorescently tagged primer and one unlabeled primer, and the PCR products were then incubated with 1 exonuclease. The unlabeled strand of the PCR product was digested by the exonuclease while the fluorescently tagged strand remained intact because the exonuclease activity on that strand was blocked by the fluorescence label at its 5′-end. After enzyme digest, the fluorescently labeled single-stranded DNA molecules were allowed to hybridize to the oligonucleotide arrays.

Another approach for generating single-stranded DNA molecules is asymmetric PCR. A two-step PCR strategy was designed in this approach. In the first step, HLA-B was amplified by PCR using two primers to generate double-stranded PCR products; in the second step, the PCR product obtained from the first amplification was amplified by PCR with only one primer, so that only one DNA strand would be amplified in this step. The single-stranded product generated in this approach had very high hybridization efficiency when applied to the oligonucleotide array.

All three methods could efficiently generate single-stranded DNA product. The strand separation method had the highest efficiency, but the high cost of the streptavidin beads made it unsuitable for large-scale testing. The exonuclease digest method and the asymmetric PCR method were both economical and efficient, but a higher concentration of single-stranded PCR product could be generated through two amplification steps in the asymmetric PCR approach. Therefore, the asymmetric PCR method was employed exclusively in our DNA sample preparation. Human genomic DNA was extracted from blood sample using standard procedures. The quality of DNA sample was tested by OD 260/280 measurement.

Exon 2 of HLA-B gene was amplified by two-step asymmetric PCR. In the first step, the PCR primers were Exon 2 5′-primer (5′-GCTCCCACTCCATGAGGTAT-3′; SEQ ID NO:71) and Exon 2 3′-primer (5′-CGGCCTCGCTCTGGTTGTAG-3′; SEQ ID NO:138). The one hundred microliter amplification reaction contained 50 mM KCl, 10 mM Tris-HCl, 1.5 mg MgCl2, 10 mg of gelatin, 20 ng of genomic DNA, 2 microMoles of each primer, 200 microMoles each of dATP, dCTP, dTTP and dGTP, and 2.5 U of Taq DNA polymerase. The amplification reaction was performed in a Perkin-Elmer Cetus 9600 thermal cycler using 35 cycles of the following profile: 94° C. for 30 seconds, 60° C. for 1 minute and 72° C. for 1 minute. The PCR mixture was then purified using a QIAGEN PCR purification kit (QIAGEN Inc., Chatsworth, Calif.) to remove the excess primers. In the second step, the PCR primer employed was a 5′ Rhodamine-labeled Exon 2 3′-primer (SEQ ID NO:138). The PCR was performed in 30 cycles using the following profile: 94° C. for 30 seconds, 60° C. for 1 minute and 72° C. for 2 minutes.

Amplification of exon 3 of HLA-B was accomplished using Exon 3 5′-primer (5′-ACCCGGTTTCATTTTCAGTTG-3′; SEQ ID NO:139) and Exon 3 3′-primer (5′-CCCACTGCCCCTGGTACC-3′; SEQ ID NO:140). The amplification reaction was performed in 35 cycles of the following profile: 94° C. for 30 seconds, 65° C. for 1 minute and 72° C. for 1 minute. To generate single-strand exon 3 product, the second PCR was performed, employing a 5′ Rhodamine-labeled 3 3′-primer (SEQ ID NO:140), in 30 cycles of the following profile: 94° C. for 1 minute, 65° C. for 1 minute and 72° C. for 2 minutes.

### EXAMPLE 4

The example illustrates the hybridization and detection of DNA samples to the HLA-B microarrays.

The fidelity of the hybridization assay is governed by the stability differences between perfectly matched and mismatched duplexes. It was desirable to find a single set of hybridization conditions that could provide a clear discrimination between matches and mismatches for all polymorphisms in each exon. In order for the melting temperature of the probe sequences to be comparable, probes were designed with careful attention to probe size, base composition, and placement of mismatched position within the hybridization sequence.

Single-stranded HLA-B, generated by asymmetric PCR using a fluorescently labeled primer, was diluted using hybridization buffer and allowed to hybridize to the oligonucleotide arrays for 3 hours. The glass slide was then washed with washing buffer at stringent conditions to remove mismatched DNA strands. Hybridization experiments designed to compare tetremethylammonium chloride (TMAC), an “isostabilization” reagent, and with buffer containing no TMAC generated the same hybridization patterns. This demonstrated that the melting temperatures of the HLA-B oligonucleotide sequences were comparable, and that a single set of conditions could be used for all HLA-B polymorphisms. For hybridization with exon 2 oligonucleotide arrays, fifty microliter solution of the single-stranded Rodamine-labeled PCR product of exon 2 in 5×SSPE, 0.5% SDS was applied to the array slide and covered with a cover glass, and incubated at 37° C. for 2 hours. The glass slide was then washed with 20 ml washing buffer (20×SSPE, 0.2% SDS) at 30° C. twice, 15 minutes each. For hybridization with exon 3 oligonucleotide arrays, fifty microliter solution of the single-stranded Rodamine-labeled PCR product of exon 3 was added to the glass slides, and incubated at 30° C. for 2 hours and then washed at room temperature twice.

After hybridization and washing process, fifty microliters of washing solution (2×SSPE, 0.2% SDS) was applied to the glass slide, and the slide was covered with a cover glass. This provides an aqueous environment for the fluorescence scanning. Positive hybridization results were detected by fluorescence scanning of the slide using a Molecular Dynamic array scanner. This instrument generated fluorescence images by scanning a laser beam over the sample surface in a raster pattern. The resulting fluorescence was monitored pixel-by-pixel through a bandpass filter. The spatial resolution of the scan was 10 microns per pixel. The fluorescence intensity of each pixel, measured by a PMT, was digitized to 16-bit precision, and the data saved to computer disk as a TIFF format file. After scanning, the fluorescence image was reconstructed from the digitized pixel intensities using image analysis software provided with the scanner. A “spot finding” program was used to verify the hybridization signals and to quantify signal intensities. A linear relationship between signal intensity and concentration of bound oligonucleotide was observed within a range of 0.5 fmole to 0.5 pmole. To calculate signal intensity, the ImageQuant software was used to sum pixel intensities within each spot image. The average value and standard deviation of pixel intensities would also been calculated and local background level subtracted. The signal-to-noise ratio was calculated as the fluorescence intensity level over background divided by the standard deviation of intensities.

### EXAMPLE 5

The example illustrates HLA-B allele assignment.

HLA-B alleles were assigned by quantitative analysis of the hybridization pattern of the DNA sample. The oligonucleotide probes were assigned into different groups. The probes in each group would hybridize with the same 20-nucleotide region in either exon 2 or exon 3, and be complementary with sequence of different alleles. When hybridized with heterozygous DNA samples, 2 oligonucleotide probes in each group might be perfectly matched with the PCR product, each matched with a different allele. When hybridized with homozygous samples, one probe in each group would be perfectly matched with the PCR product. A theoretical hybridization pattern was generated for each allele by counting the probes matched with its sequence.

All hybridization signal intensities were quantified and ranked from the highest to the lowest in each probe group. When hybridized with homozygous samples, only one probe in each group produces positive signal as the perfect match. In samples heterozygous for two different HLA-B alleles, two probes with highest signal intensities in each group are selected as positive unless only one positive signal was shown in that group. Assignment of the alleles was accomplished by comparing the detected hybridization pattern, with the theoretical patterns of all known HLA-B alleles.

A blinded equivalency study of 60 different DNA samples that were previously typed using sequencing methods was conducted. HLA-B allele assignments were made by an independent observer who had no knowledge of the genotypes of the samples. The same alleles were assigned with the array method as were previously determined by sequencing for all 120 alleles in the study group.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of HLA-B tissue typing, said method comprising:
(a) amplifying exons 2 and 3 from a genomic sample of tissue using labeled primers and an asymmetric PCR method to form a labeled, single-stranded DNA sample;
(b) contacting said labeled, single-stranded DNA sample with 4 an array of HLA-B oligonucleotide probes under hybridization conditions; and
(c) detecting hybridization for said DNA sample and assigning an HLA-B allele type by analysis of said hybridization.

2. A method in accordance with claim 1, wherein said HLA-B oligonucleotide probes are attached to one or more solid supports selected from the group consisting of plates, slides, capillaries, spheres, particles, gels and films.

3. A method in accordance with claim 1, wherein said HLA-B oligonucleotide probes are HLA-B exon 2 and exon 3 oligonucleotide probes.

4. A method in accordance with claim 1, wherein the HLA-B oligonucleotide probes each have from 17 to 23 nucleotides.

5. A method in accordance with claim 2, wherein the HLA-B oligonucleotide probes are present on said solid support at a surface density of from about 250 to about 450 angstrom²/molecule.

6. A method in accordance with claim 1, wherein the HLA-B oligonucleotide probes are present on said solid support at a surface density of from about 325 to about 375 angstrom²/molecule.

7. A method in accordance with claim 1, wherein said array is on a glass slide or plate.

8. A method in accordance with claim 1, wherein said array is on beads.


SEQUENCE LISTING (141 sequences)

SEQ ID NO: 1 - DNA, 20 bp, Artificial Sequence
  HLA-B PCR probe A1, exon 2, location 5-24
tcacaccctccagagcatgt

SEQ ID NO: 2 - DNA, 20 bp, Artificial Sequence
  HLA-B PCR probe A2, exon 2, location 5-24
tcacatcatccagaggatgt

SEQ ID NO: 3 - DNA, 20 bp, Artificial Sequence
  HLA-B PCR probe A3, exon 2, location 5-24
tcacacttggcagacgatgt

SEQ ID NO: 4 - DNA, 20 bp, Artificial Sequence
  HLA-B PCR probe A4, exon 2, location 5-24
tcacaccctccagtggatgt

SEQ ID NO: 5 - DNA, 20 bp, Artificial Sequence
  HLA-B PCR probe A5, exon 2, location 5-24
tcacacttggcagaggatgt

SEQ ID NO: 6 - DNA, 20 bp, Artificial Sequence
  HLA-B PCR probe A6, exon 2, location 5-24
tcacaccctccagacgatgt

SEQ ID NO: 7 - DNA, 20 bp, Artificial Sequence
  HLA-B PCR probe A7, exon 2, location 5-24
tcacaccctccagaatatgt

SEQ ID NO: 8 - DNA, 20 bp, Artificial Sequence
  HLA-B PCR probe A8, exon 2, location 5-24
tcacatcatccagagcatgt

SEQ ID NO: 9 - DNA, 20 bp, Artificial Sequence
  HLA-B PCR probe A9, exon 2, location 5-24
tcacaccatccagaggatgt

SEQ ID NO: 10 - DNA, 20 bp, Artificial Sequence
  HLA-B PCR probe A10, exon 2, location 5-24
tcacatcatccaggtgatgt

SEQ ID NO: 11 - DNA, 20 bp, Artificial Sequence
  HLA-B PCR probe A11, exon 2, location 5-24
tcacaccctccagaggatgt

SEQ ID NO: 12 - DNA, 20 bp, Artificial Sequence
  HLA-B PCR probe B1, exon 2, location 21-40
atgtacggctgcgacgtggg

SEQ ID NO: 13 - DNA, 20 bp, Artificial Sequence
  HLA-B PCR probe B2, exon 2, location 21-40
atgtatggctgcgacctggg

SEQ ID NO: 14 - DNA, 20 bp, Artificial Sequence
  HLA-B PCR probe B3, exon 2, location 21-40
atgtttggctgcgacgtggg

SEQ ID NO: 15 - DNA, 20 bp, Artificial Sequence
  HLA-B PCR probe B4, exon 2, location 21-40
atgtaaggctgcgacgtggg

SEQ ID NO: 16 - DNA, 20 bp, Artificial Sequence
  HLA-B PCR probe B5, exon 2, location 21-40
atgtctggctgcgacgtggg

SEQ ID NO: 17 - DNA, 20 bp, Artificial Sequence
  HLA-B PCR probe B6, exon 2, location 21-40
atgtacggctgcgacctggg

SEQ ID NO: 18 - DNA, 20 bp, Artificial Sequence
  HLA-B PCR probe B7, exon 2, location 21-40
atgtttggctgcgacctggg

SEQ ID NO: 19 - DNA, 20 bp, Artificial Sequence
  HLA-B PCR probe B8, exon 2, location 21-40
atgtatggctgcgacatggg

SEQ ID NO: 20 - DNA, 20 bp, Artificial Sequence
  HLA-B PCR probe B9, exon 2, location 21-40
atgtatggctgcgacgtggg

SEQ ID NO: 21 - DNA, 20 bp, Artificial Sequence
  HLA-B PCR probe C1, exon 2, location 41-60
gcccgacgggcgcctcctcc

SEQ ID NO: 22 - DNA, 20 bp, Artificial Sequence
  HLA-B PCR probe C2, exon 2, location 41-60
gcccgacgggcgcttcctcc

SEQ ID NO: 23 - DNA, 20 bp, Artificial Sequence
  HLA-B PCR probe C3, exon 2, location 41-60
gccggacgggcgcctcctcc

SEQ ID NO: 24 - DNA, 20 bp, Artificial Sequence
  HLA-B PCR probe D1, exon 2, location 61-80
gcgggcatgaccagtacgcc

SEQ ID NO: 25 - DNA, 20 bp, Artificial Sequence
  HLA-B PCR probe D2, exon 2, location 61-80
gcgggcataaccagtacgcc

SEQ ID NO: 26 - DNA, 20 bp, Artificial Sequence
  HLA-B PCR probe D3, exon 2, location 61-80
gcgggcataaccagttagcc

SEQ ID NO: 27 - DNA, 20 bp, Artificial Sequence
  HLA-B PCR probe D4, exon 2, location 61-80
gcgggtataaccagttcgcc

SEQ ID NO: 28 - DNA, 20 bp, Artificial Sequence
  HLA-B PCR probe D5, exon 2, location 61-80
gcgggcatgaccagtccgcc

SEQ ID NO: 29 - DNA, 20 bp, Artificial Sequence
  HLA-B PCR probe D6, exon 2, location 61-80
gcgggtatgaccagtccgcc

SEQ ID NO: 30 - DNA, 20 bp, Artificial Sequence
  HLA-B PCR probe D7, exon 2, location 61-80
gcgggtaccaccaggacgcc

SEQ ID NO: 31 - DNA, 20 bp, Artificial Sequence
  HLA-B PCR probe D8, exon 2, location 61-80
gcgggcatgaccagttcgcc

SEQ ID NO: 32 - DNA, 20 bp, Artificial Sequence
  HLA-B PCR probe D9, exon 2, location 61-80
gcgggcataaccagttcgcc

SEQ ID NO: 33 - DNA, 20 bp, Artificial Sequence
  HLA-B PCR probe D10, exon 2, location 61-80
gcgggtatgaccaggacgcc

SEQ ID NO: 34 - DNA, 20 bp, Artificial Sequence
  HLA-B PCR probe D11, exon 2, location 61-80
gcgggtataaccagttagcc

SEQ ID NO: 35 - DNA, 20 bp, Artificial Sequence
  HLA-B PCR probe D12, exon 2, location 61-80
gcgggtatgaccagtacgcc

SEQ ID NO: 36 - DNA, 20 bp, Artificial Sequence
  HLA-B PCR probe E1, exon 2, location 81-100
tacgacggcaaagattacat

SEQ ID NO: 37 - DNA, 20 bp, Artificial Sequence
  HLA-B PCR probe E2, exon 2, location 81-100
tacgacggcaaggattacat

SEQ ID NO: 38 - DNA, 20 bp, Artificial Sequence
  HLA-B PCR probe F1, exon 2, location 111-130
gaggacctgagctcctggac

SEQ ID NO: 39 - DNA, 20 bp, Artificial Sequence
  HLA-B PCR probe F2, exon 2, location 111-130
gaggacctgcgctcctggac

SEQ ID NO: 40 - DNA, 20 bp, Artificial Sequence
  HLA-B PCR probe G1, exon 2, location 131-150
cgccgcggacacggcggctc

SEQ ID NO: 41 - DNA, 20 bp, Artificial Sequence
  HLA-B PCR probe G2, exon 2, location 131-150
cgcggcggacaccgcggctc

SEQ ID NO: 42 - DNA, 20 bp, Artificial Sequence
  HLA-B PCR probe G3, exon 2, location 131-150
cgccgcggacaaggcggctc

SEQ ID NO: 43 - DNA, 20 bp, Artificial Sequence
  HLA-B PCR probe G4, exon 2, location 131-150
cgccgcggacacggcagctc

SEQ ID NO: 44 - DNA, 20 bp, Artificial Sequence
  HLA-B PCR probe G5, exon 2, location 131-150
cgccgcggacaccgcggctc

SEQ ID NO: 45 - DNA, 20 bp, Artificial Sequence
  HLA-B PCR probe G6, exon 2, location 131-150
cgcggcggacacggcggctc

SEQ ID NO: 46 - DNA, 20 bp, Artificial Sequence
  HLA-B PCR probe H1, exon 2, location 151-170
agatcacccagctcaagtgg

SEQ ID NO: 47 - DNA, 20 bp, Artificial Sequence
  HLA-B PCR probe H2, exon 2, location 151-170
agatctcccagcgcaagttg

SEQ ID NO: 48 - DNA, 20 bp, Artificial Sequence
  HLA-B PCR probe H3, exon 2, location 151-170
agatcacccagcgcaagtgg

SEQ ID NO: 49 - DNA, 20 bp, Artificial Sequence
  HLA-B PCR probe I1, exon 2, location 171-190
gaggcggcccgtgaggcgga

SEQ ID NO: 50 - DNA, 20 bp, Artificial Sequence
  HLA-B PCR probe I2, exon 2, location 171-190
gaggcggcccgtgtggcgga

SEQ ID NO: 51 - DNA, 20 bp, Artificial Sequence
  HLA-B PCR probe J1, exon 2, location 191-210
gcagcggagagcctacctgg

SEQ ID NO: 52 - DNA, 20 bp, Artificial Sequence
  HLA-B PCR probe J2, exon 2, location 191-210
gcaggacagagcctacctgg

SEQ ID NO: 53 - DNA, 20 bp, Artificial Sequence
  HLA-B PCR probe J3, exon 2, location 191-210
gcagtggagagcctacctgg

SEQ ID NO: 54 - DNA, 20 bp, Artificial Sequence
  HLA-B PCR probe J4, exon 2, location 191-210
gcagctgagaacctacctgg

SEQ ID NO: 55 - DNA, 20 bp, Artificial Sequence
  HLA-B PCR probe J5, exon 2, location 191-210
gcagcggagaacctacctgg

SEQ ID NO: 56 - DNA, 20 bp, Artificial Sequence
  HLA-B PCR probe J6, exon 2, location 191-210
gcagctgagagcctacctgg

SEQ ID NO: 57 - DNA, 20 bp, Artificial Sequence
  HLA-B PCR probe K1, exon 2, location 211-230
agggcgagtgcgtggagtgg

SEQ ID NO: 58 - DNA, 20 bp, Artificial Sequence
  HLA-B PCR probe K2, exon 2, location 211-230
agggcctgtgcgtggagtgg

SEQ ID NO: 59 - DNA, 20 bp, Artificial Sequence
  HLA-B PCR probe K3, exon 2, location 211-230
agggcctgtgcgtggacggg

SEQ ID NO: 60 - DNA, 20 bp, Artificial Sequence
  HLA-B PCR probe K4, exon 2, location 211-230
agggcctgtgcgtggagtcg

SEQ ID NO: 61 - DNA, 20 bp, Artificial Sequence
  HLA-B PCR probe K5, exon 2, location 211-230
agggcacgtgcgtggagtcg

SEQ ID NO: 62 - DNA, 20 bp, Artificial Sequence
  HLA-B PCR probe K6, exon 2, location 211-230
agggcctgtgcgtggagggg

SEQ ID NO: 63 - DNA, 20 bp, Artificial Sequence
  HLA-B PCR probe K7, exon 2, location 211-230
aggacctgtgcgtggagtcg

SEQ ID NO: 64 - DNA, 20 bp, Artificial Sequence
  HLA-B PCR probe K8, exon 2, location 211-230
agggcacgtgcgtggagtgg

SEQ ID NO: 65 - DNA, 20 bp, Artificial Sequence
  HLA-B PCR probe L1, exon 2, location 231-250
ctccgcagacacctggagaa

SEQ ID NO: 66 - DNA, 20 bp, Artificial Sequence
  HLA-B PCR probe L2, exon 2, location 231-250
ctccgcagatacctggagaa

SEQ ID NO: 67 - DNA, 20 bp, Artificial Sequence
  HLA-B PCR probe M1, exon 2, location 257-276
ggacaagctggagcgcgctg

SEQ ID NO: 68 - DNA, 20 bp, Artificial Sequence
  HLA-B PCR probe M2, exon 2, location 257-276
ggacacgctggagcgcgcgg

SEQ ID NO: 69 - DNA, 20 bp, Artificial Sequence
  HLA-B PCR probe M3, exon 2, location 257-276
ggagacgctgcagcgcgcgg

SEQ ID NO: 70 - DNA, 20 bp, Artificial Sequence
  HLA-B PCR probe A1, exon 3, location 01-20
gctcccacttcatgaggtat

SEQ ID NO: 71 - DNA, 20 bp, Artificial Sequence
  HLA-B PCR probe A2, exon 3, location 01-20, two-step asymmetric PCR HLA-B Exon 2 5′ primer
gctcccactccatgaggtat

SEQ ID NO: 72 - DNA, 20 bp, Artificial Sequence
  HLA-B PCR probe B1, exon 3, location 21-40
ttctacacctccgtgtcccg

SEQ ID NO: 73 - DNA, 20 bp, Artificial Sequence
  HLA-B PCR probe B2, exon 3, location 21-40
ttctacaccgccatgtcccg

SEQ ID NO: 74 - DNA, 20 bp, Artificial Sequence
  HLA-B PCR probe B3, exon 3, location 21-40
ttcgacaccgccatgtcccg

SEQ ID NO: 75 - DNA, 20 bp, Artificial Sequence
  HLA-B PCR probe B4, exon 3, location 21-40
ttccacacctccgtgtcccg

SEQ ID NO: 76 - DNA, 20 bp, Artificial Sequence
  HLA-B PCR probe B5, exon 3, location 21-40
ttccacaccgccatgtcccg

SEQ ID NO: 77 - DNA, 20 bp, Artificial Sequence
  HLA-B PCR probe B6, exon 3, location 21-40
ttctacaccgctatgtcccg

SEQ ID NO: 78 - DNA, 20 bp, Artificial Sequence
  HLA-B PCR probe B7, exon 3, location 21-40
ttctacaccgccgtgtcccg

SEQ ID NO: 79 - DNA, 20 bp, Artificial Sequence
  HLA-B PCR probe C1, exon 3, location 41-60
gcccgtccgcggggagcccc

SEQ ID NO: 80 - DNA, 20 bp, Artificial Sequence
  HLA-B PCR probe C2, exon 3, location 41-60
gcctggccgcggggagcccc

SEQ ID NO: 81 - DNA, 20 bp, Artificial Sequence
  HLA-B PCR probe C3, exon 3, location 41-60
gcccggccgcggggagcccc

SEQ ID NO: 82 - DNA, 20 bp, Artificial Sequence
  HLA-B PCR probe D1, exon 3, location 61-80
gcttcatctcagtgggctac

SEQ ID NO: 83 - DNA, 20 bp, Artificial Sequence
  HLA-B PCR probe D2, exon 3, location 61-80
gcttcatcaccgtgggctac

SEQ ID NO: 84 - DNA, 20 bp, Artificial Sequence
  HLA-B PCR probe D3, exon 3, location 61-80
gcttcattgcagtgggctac

SEQ ID NO: 85 - DNA, 20 bp, Artificial Sequence
  HLA-B PCR probe D4, exon 3, location 61-80
gcttcatcgcagtgggctac

SEQ ID NO: 86 - DNA, 20 bp, Artificial Sequence
  HLA-B PCR probe E1, exon 3, location 81-100
gtggacgacacccagttcgt

SEQ ID NO: 87 - DNA, 20 bp, Artificial Sequence
  HLA-B PCR probe E2, exon 3, location 81-100
gtggacggcacccagttcgt

SEQ ID NO: 88 - DNA, 20 bp, Artificial Sequence
  HLA-B PCR probe E3, exon 3, location 81-100
gtggacgacacgctgttcgt

SEQ ID NO: 89 - DNA, 20 bp, Artificial Sequence
  HLA-B PCR probe E4, exon 3, location 81-100
gtggacgacacgcagttcgt

SEQ ID NO: 90 - DNA, 20 bp, Artificial Sequence
  HLA-B PCR probe F1, exon 3, location 111-130
agcgacgccacgagtccgag

SEQ ID NO: 91 - DNA, 20 bp, Artificial Sequence
  HLA-B PCR probe F2, exon 3, location 111-130
agcgacgccgcgagtccgag

SEQ ID NO: 92 - DNA, 20 bp, Artificial Sequence
  HLA-B PCR probe G1, exon 3, location 95-115
ttcgtgcggttcgacagcga

SEQ ID NO: 93 - DNA, 20 bp, Artificial Sequence
  HLA-B PCR probe G2, exon 3, location 96-115
ttcgtgaggttcgacagcga

SEQ ID NO: 94 - DNA, 20 bp, Artificial Sequence
  HLA-B PCR probe H1, exon 3, location 121-140
cgagtccgagagaggagccg

SEQ ID NO: 95 - DNA, 20 bp, Artificial Sequence
  HLA-B PCR probe H2, exon 3, location 121-140
cgagtccgaggatggcgccc

SEQ ID NO: 96 - DNA, 20 bp, Artificial Sequence
  HLA-B PCR probe H3, exon 3, location 121-140
cgagtccgaggacggagccc

SEQ ID NO: 97 - DNA, 20 bp, Artificial Sequence
  HLA-B PCR probe H4, exon 3, location 121-140
cgagtccgaggaaggagccg

SEQ ID NO: 98 - DNA, 20 bp, Artificial Sequence
  HLA-B PCR probe I1, exon 3, location 141-160
cgggcgccatggatagagca

SEQ ID NO: 99 - DNA, 20 bp, Artificial Sequence
  HLA-B PCR probe I2, exon 3, location 141-160
cgggcgccgtgggtggagca

SEQ ID NO: 100 - DNA, 20 bp, Artificial Sequence
  HLA-B PCR probe I3, exon 3, location 141-160
cgggcgccgtggatagagca

SEQ ID NO: 101 - DNA, 20 bp, Artificial Sequence
  HLA-B PCR probe J1, exon 3, location 161-180
ggaggggccggaatattggg

SEQ ID NO: 102 - DNA, 20 bp, Artificial Sequence
  HLA-B PCR probe J2, exon 3, location 161-180
agaggggccggagtattggg

SEQ ID NO: 103 - DNA, 20 bp, Artificial Sequence
  HLA-B PCR probe J3, exon 3, location 161-180
ggaggggccggagcattggg

SEQ ID NO: 104 - DNA, 20 bp, Artificial Sequence
  HLA-B PCR probe J4, exon 3, location 161-180
ggaggggccggagtattggg

SEQ ID NO: 105 - DNA, 20 bp, Artificial Sequence
  HLA-B PCR probe K1, exon 3, location 181-200
accggaacacacagatctac

SEQ ID NO: 106 - DNA, 20 bp, Artificial Sequence
  HLA-B PCR probe K2, exon 3, location 181-200
accggaacacacagatcttc

SEQ ID NO: 107 - DNA, 20 bp, Artificial Sequence
  HLA-B PCR probe K3, exon 3, location 181-200
accgggagacacagatctcc

SEQ ID NO: 108 - DNA, 20 bp, Artificial Sequence
  HLA-B PCR probe K4, exon 3, location 181-200
accggaacacacagatctgc

SEQ ID NO: 109 - DNA, 20 bp, Artificial Sequence
  HLA-B PCR probe K5, exon 3, location 181-200
accggaacacacagatctcc

SEQ ID NO: 110 - DNA, 20 bp, Artificial Sequence
  HLA-B PCR probe K6, exon 3, location 181-200
accgggagacacggaacatg

SEQ ID NO: 111 - DNA, 20 bp, Artificial Sequence
  HLA-B PCR probe K7, exon 3, location 181-200
accgggatacacagatctcc

SEQ ID NO: 112 - DNA, 20 bp, Artificial Sequence
  HLA-B PCR probe K8, exon 3, location 181-200
accgggagacacagatctgc

SEQ ID NO: 113 - DNA, 20 bp, Artificial Sequence
  HLA-B PCR probe K9, exon 3, location 181-200
accgggagacacagaagtac

SEQ ID NO: 114 - DNA, 20 bp, Artificial Sequence
  HLA-B PCR probe K10, exon 3, location 181-200
acggggagacacggaacatg

SEQ ID NO: 115 - DNA, 20 bp, Artificial Sequence
  HLA-B PCR probe K11, exon 3, location 181-200
accgggagacacagatcttc

SEQ ID NO: 116 - DNA, 20 bp, Artificial Sequence
  HLA-B PCR probe L1, exon 3, location 201-220
aaggcccaggcacagactga

SEQ ID NO: 117 - DNA, 20 bp, Artificial Sequence
  HLA-B PCR probe L2, exon 3, location 201-220
aagaccaacacacagactta

SEQ ID NO: 118 - DNA, 20 bp, Artificial Sequence
  HLA-B PCR probe L3, exon 3, location 201-220
aaggcctccgcgcagactta

SEQ ID NO: 119 - DNA, 20 bp, Artificial Sequence
  HLA-B PCR probe L4, exon 3, location 201-220
aaggccaaggcacagactta

SEQ ID NO: 120 - DNA, 20 bp, Artificial Sequence
  HLA-B PCR probe L5, exon 3, location 201-220
aaggccaaggcacagactga

SEQ ID NO: 121 - DNA, 20 bp, Artificial Sequence
  HLA-B PCR probe L6, exon 3, location 201-220
aagcgccaggcacagactga

SEQ ID NO: 122 - DNA, 20 bp, Artificial Sequence
  HLA-B PCR probe L7, exon 3, location 201-220
aagaccaacacacagactga

SEQ ID NO: 123 - DNA, 20 bp, Artificial Sequence
  HLA-B PCR probe M1, exon 3, location 216-235
actgaccgagagagcctgcg

SEQ ID NO: 124 - DNA, 20 bp, Artificial Sequence
  HLA-B PCR probe M2, exon 3, location 216-235
acttaccgagagaacctgcg

SEQ ID NO: 125 - DNA, 20 bp, Artificial Sequence
  HLA-B PCR probe M3, exon 3, location 216-235
acttaccgagagagcctgcg

SEQ ID NO: 126 - DNA, 20 bp, Artificial Sequence
  HLA-B PCR probe M4, exon 3, location 216-235
actgaccgagaggacctgcg

SEQ ID NO: 127 - DNA, 20 bp, Artificial Sequence
  HLA-B PCR probe M5, exon 3, location 216-235
acttaccgagaggacctgcg

SEQ ID NO: 128 - DNA, 20 bp, Artificial Sequence
  HLA-B PCR probe M6, exon 3, location 216-235
actgaccgagtgagcctgcg

SEQ ID NO: 129 - DNA, 20 bp, Artificial Sequence
  HLA-B PCR probe M7, exon 3, location 216-235
actgaccgagtgggcctgcg

SEQ ID NO: 130 - DNA, 20 bp, Artificial Sequence
  HLA-B PCR probe M8, exon 3, location 216-235
actgaccgagagaacctgcg

SEQ ID NO: 131 - DNA, 20 bp, Artificial Sequence
  HLA-B PCR probe N1, exon 3, location 231-250
ctgcgcaccgcgctccgcta

SEQ ID NO: 132 - DNA, 20 bp, Artificial Sequence
  HLA-B PCR probe N2, exon 3, location 231-250
ctgcggatcgcgctccgcta

SEQ ID NO: 133 - DNA, 20 bp, Artificial Sequence
  HLA-B PCR probe N3, exon 3, location 231-250
ctgcggaccctgctccgcta

SEQ ID NO: 134 - DNA, 20 bp, Artificial Sequence
  HLA-B PCR probe N4, exon 3, location 231-250
ctgcggaacctgctccgcta

SEQ ID NO: 135 - DNA, 20 bp, Artificial Sequence
  HLA-B PCR probe N5, exon 3, location 231-250
ctgcggaacctgcgcggcta

SEQ ID NO: 136 - DNA, 20 bp, Artificial Sequence
  HLA-B PCR probe O1, exon 3, location 251-270
ctacaaccagagcgaggacg

SEQ ID NO: 137 - DNA, 20 bp, Artificial Sequence
  HLA-B PCR probe O2, exon 3, location 251-270
ctacaaccagagcgaggccg

SEQ ID NO: 138 - DNA, 20 bp, Artificial Sequence
  two-step asymmetric PCR HLA-B Exon 2 3′ primer
cggcctcgctctggttgtag

SEQ ID NO: 139 - DNA, 21 bp, Artificial Sequence
  PCR amplification HLA-B Exon 3 5′ primer
acccggtttcattttcagttg

SEQ ID NO: 140 - DNA, 18 bp, Artificial Sequence
  PCR amplification HLA-B Exon 3 3′ primer
cccactgcccctggtacc

SEQ ID NO: 141 - DNA, 15 bp, Artificial Sequence
  15-mer poly dT spacing group
ttttttttttttttt